(19) United States
(12) Patent Application Publication
(10) Pub. No.: US 2023/0398240 A1
(43) Pub. Date: Dec. 14, 2023

(54) THERAPEUTIC RADIOLABELLED CONJUGATES AND THEIR USE IN THERAPY

(71) Applicants: CENTENARY INSTITUTE OF CANCER MEDICINE AND CELL BIOLOGY, Camperdown (AU); UNIVERSITY OF SYDNEY, Sydney (AU)

(72) Inventors: Philip HOGG, Malabar (AU); Ivan HO SHON, Strathfield (AU)

(21) Appl. No.: 18/299,304

(22) Filed: Apr. 12, 2023

Related U.S. Application Data
(63) Continuation of application No. PCT/AU2021/051203, filed on Oct. 14, 2021.

(30) Foreign Application Priority Data
Oct. 14, 2020 (AU) 2020903727

(51) Int. Cl. A61K 51/04; A61P 35/00

(52) U.S. Cl. CPC A61K 51/0497; A61P 35/00

(57) ABSTRACT

The invention relates to compounds according to Formula (I) wherein A is —As(OH)₂ or an arsenoxide equivalent group; each of R₁, R₂, R₃ and R₄ is independently selected from H, X, OH, NH₂, CO, SCN, —CH₂NH, —NHCOCH₃, —NHCOCH₂X or NO, and X is a halogen; R₅ is —NHCH₂COOH, OH or OR₆, wherein R6 is a C₁₋₅ straight or branched alkyl group; and Z is a therapeutic radioisotope, or a pharmaceutically acceptable salt, ester, prodrug or solvate thereof, uses of said compounds, and methods of preparing said compounds. The invention also relates to therapeutic methods utilizing said compounds. The invention further relates to processes for preparing compounds according to Formula (I) wherein Z is a radioisotope.

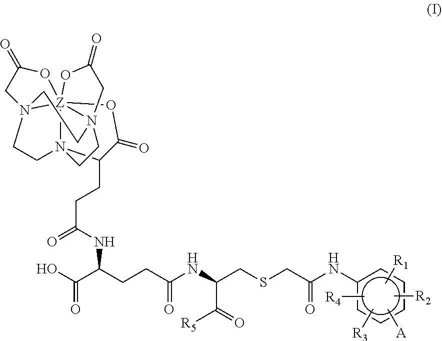

### CROSS-REFERENCE TO RELATED APPLICATIONS

This application is a continuation of International Application No. PCT/AU2021/051203, filed on Oct. 14, 2021, which claims priority to Australian Patent Application No. 2020903727, filed on Oct. 14, 2020. The entire contents of these applications are incorporated by reference herein, in their entirety.

### TECHNICAL FIELD

The present invention broadly relates to a radiolabelled conjugate according to Formula (I) defined herein. The present invention further relates to the use of such radiolabelled conjugates in treatment of neoplastic conditions, and methods of producing such radiolabelled conjugates.

### BACKGROUND OF THE INVENTION

Cancer is responsible for about 1 in 6 deaths globally and the economic cost is in the trillions of dollars annually. Tumours result from an imbalance between rates of cellular proliferation and survival in a tissue, while successful treatment controls tumour growth by inhibiting tumour cell proliferation and/or promoting tumour cell death.

Chemotherapy, radiotherapy and immunotherapy are the mainstay of cancer therapies and are effective in many cases. When cancer is localised, it is amenable to potentially curative treatments such as surgery or synergistic combinations such as chemo-radiotherapy. However, once disease is disseminated, systemic therapies such as chemotherapy, targeted therapies or immunotherapy are required. Whilst the addition of external beam radiotherapy may be synergistic in patients with disseminated disease, it is often not possible to treat all sites of disease due to toxicity, and thus radiotherapy is reserved for palliative treatment of symptomatic sites of disease. Curative treatments remain in the minority for most cancer types. In addition, the efficacy of these treatment strategies is limited by the heterogeneity of the tumour, as certain populations of cancer cells become resistant to therapy.

There has been renewed interest in theranostics to improve curative rates for solid tumours. A theranostic employs a tumour marker to deliver a therapeutic isotope to the tumour. Theranostic approaches have proved successful in niche applications such as neuroendocrine tumours (Strosberg J, El-Haddad G, Wolin E, et al. Phase 3 Trial of (177)Lu-Dotatate for Midgut Neuroendocrine Tumors. N Engl J Med. 2017; 376(2):125-135) and prostate carcinoma (von Eyben F E, Roviello G, Kiljunen T, et al. Third-line treatment and (177)Lu-PSMA radioligand therapy of metastatic castration-resistant prostate cancer: a systematic review. Eur J Nucl Med Mol Imaging. 2018; 45(3):496-508), wherein the tumour markers are somatostatin receptor and prostate specific membrane antigen, respectively. However, theranostic approaches have traditionally been limited to niche applications and select tumour groups.

It remains desirable to provide effective targeted treatments for cancers.

### SUMMARY OF THE INVENTION

According to a first aspect, the present disclosure provides a compound according to Formula (I)

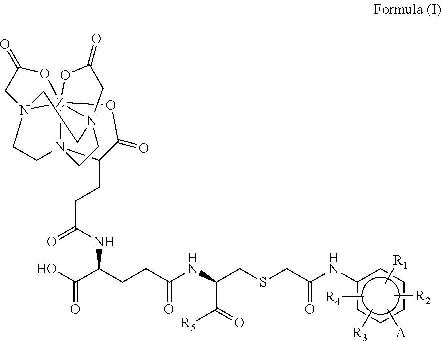

wherein A is —As(OH)₂ or an arsenoxide equivalent group; each of R₁, R₂, R₃ and R₄ is independently selected from H, X, OH, NH₂, CO, SCN, —CH₂NH, —NHCOCH₃, —NHCOCH₂X or NO, and X is a halogen; R₅ is —NHCH₂COOH, OH or OR₆, wherein R₆ is a C₁₋₅ straight or branched alkyl group; and Z is a therapeutic radioisotope, or a pharmaceutically acceptable salt, ester, prodrug or solvate thereof.

Compounds according to the present disclosure are useful for providing an effective, targeted treatment for cancers and tumours by delivering a therapeutic radioisotope selectively to areas of high cell death, such as tumours, to further enhance cell death; radiation from the radioisotope causes cell death in viable adjacent tumour cells. This induced cell death may then attract further binding of compound of the present disclosure, leading to an amplifying effect on efficacy of the treatment. Compounds of the present disclosure may optionally be administered multiple times to take advantage of such an amplifying effect. Further, combination with existing cancer therapies, such as chemotherapy, provides a highly effective positive-feedback mechanism for cancer treatment; treatment such as chemotherapy induces cell death in tumours, which in turn attracts more compound of the present disclosure, which induces further cell death in adjacent cells, which may attract further compound of the present disclosure.

In some embodiments, each of R₁, R₂, R₃ and R₄ are H. In some embodiments, R₅ is —NHCH₂COOH. In some embodiments, the compound is a compound according to Formula (Ia)

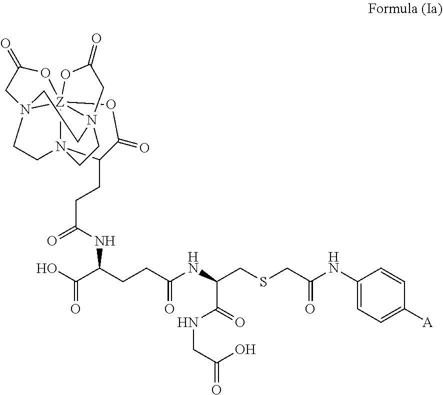

wherein A and Z are as defined above, or a pharmaceutically acceptable salt, ester, prodrug or solvate thereof.

In some embodiments, wherein Z is ¹⁷⁷Lu, ⁶⁴Cu, ⁶⁷Cu, 9° Y ¹⁸⁶Re or ¹⁸⁸Re. In some particular embodiments, Z is ¹⁷⁷Lu, ⁶⁷Cu or ⁹⁰Y. In some embodiments, Z is ¹⁷⁷Lu, ⁶⁷Cu or ⁶⁴Cu. In some particular embodiments, Z is ¹⁷⁷Lu or ⁶⁷Cu. Such isotopes are known to be useful in cancer and/or tumour therapies by inducing cell death, and Lu and Cu have been demonstrated herein to be readily incorporated into the compounds of the present application, with high efficiency and resulting stability of the compounds. The aforementioned isotopes also emit imageable emissions and thus are also useful as imaging isotopes, to determine where and how much therapeutic radiation has been delivered within a subject. Such imaging may take place, for example, by way of positron emission tomography. The use of such isotopes thus provides a theranostic compound which can be used both for therapy and for imaging, for imaging delivery of the therapeutic to areas of cell death.

In some embodiments, Z is not ⁶⁴Cu.

A particularly preferred compound is a compound according to Formula (I) wherein Z is ¹⁷⁷Lu or ⁶⁷Cu, R₁-R₄ are H, R₅ is —NHCH₂COOH, and A is As(OH)₂. Such embodiments are readily synthesised, being synthesised from readily available and affordable starting materials, and incorporate therapeutic isotopes useful for cancer and/or tumour treatment.

The present disclosure provides the compounds according to the first aspect for use in therapy. In particular, the compounds exert a therapeutic effect by inducing cell death. In some embodiments, the compounds are for use in the treatment of a neoplastic condition. In some embodiments, the neoplastic condition is a tumour. In some embodiments, the tumour is a solid tumour. In some embodiments, the neoplastic condition is cancer. In particular embodiments, the compound treats the neoplastic condition by inducing cell death.

According to a second aspect, the present disclosure provides a pharmaceutical composition comprising the compound according to the first aspect together with a pharmaceutically acceptable carrier, excipient, diluent, vehicle and/or adjuvant.

According to a third aspect, the present disclosure provides a method of treating a neoplastic condition in a subject comprising administering an effective amount of a compound according to the first aspect or a pharmaceutical composition according to the second aspect to said subject. In some embodiments, the neoplastic condition is a tumour. In some embodiments, the tumour is a solid tumour. In some embodiments, the neoplastic condition is cancer.

In some embodiments, the compound according to the first aspect or the pharmaceutical composition according to the second aspect is administered intravenously.

In some embodiments, the method of the first aspect comprises administering an effective amount of a compound according to the first aspect or a pharmaceutical composition according to the second aspect to said subject in two or more cycles, wherein efficacy of the administration against the neoplastic condition increases across the two or more cycles.

In some embodiments, the method of the third aspect comprises:
a) carrying out a treatment for said neoplastic condition on a subject other than administering an effective amount of a compound according to the first aspect or a pharmaceutical composition according to the second aspect to said subject; and
b) administering an effective amount of a compound according to the first aspect or a pharmaceutical composition according to the second aspect to said subject.

In some embodiments, the treatment carried out in step a) is chemotherapy, radiotherapy, immunotherapy and/or targeted therapy.

In some embodiments, step a) is carried out concurrently with step b), or step b) is carried out after step a).

In some embodiments, step b) is carried out for two or more cycles. In some such embodiments, the efficacy of step b) against the neoplastic condition increases across the two or more cycles. In some embodiments, both steps a) and b) are carried out for two or more cycles.

In particular embodiments, the compound according to the first aspect treats the neoplastic condition by inducing cell death.

According to a fourth aspect, the present disclosure provides a method of inducing cell death in a subject, comprising administering a compound according to the first aspect or a pharmaceutical composition according to the second aspect to a subject. In some embodiments, the compound according to the first aspect or the pharmaceutical composition according to the second aspect is administered to the subject in multiple cycles, wherein the amount of cell death induced increases across the multiple cycles.

According to a fifth aspect, the present disclosure provides use of a compound according to the first aspect in the manufacture of a medicament for the treatment of a neoplastic condition. In some embodiments, the neoplastic condition is a tumour. In some embodiments, the tumour is a solid tumour. In some embodiments, the neoplastic condition is cancer. In some embodiments, the treatment comprises a method according to the third aspect. In particular embodiments, the medicament treats the neoplastic condition by inducing cell death.

According to a sixth aspect, the present disclosure provides a process for preparing a compound according to the first aspect, comprising adding the therapeutic radioisotope to a compound according to Formula (II)

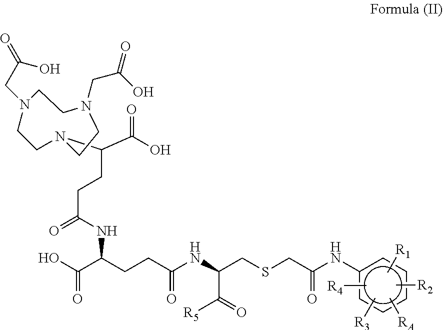

wherein A is —As(OH)₂ or an arsenoxide equivalent group;
each of R₁, R₂, R₃ and R₄ is independently selected from H, X, OH, NH₂, CO, SCN, —CH₂NH, —NHCOCH₃, —NHCOCH₂X or NO, and X is a halogen;
R₅ is —NHCH₂COOH, OH or OR₆, wherein R₆ is a C₁₋₅ straight or branched alkyl group; or a pharmaceutically acceptable salt, ester, prodrug or solvate thereof.

In some embodiments, each of R₁, R₂, R₃ and R₄ are H. In some embodiments, R₅ is —NHCH₂COOH. In some embodiments, the compound according to Formula (II) is a compound according to Formula (IIa)

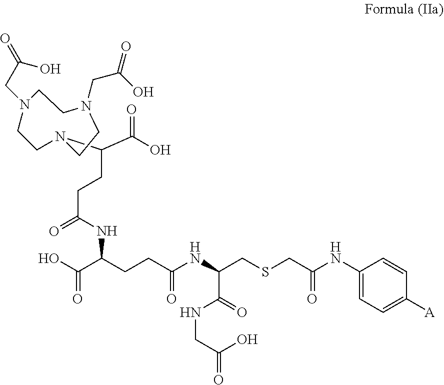

wherein A is as defined in formula (II),
or a pharmaceutically acceptable salt, ester, prodrug or solvate thereof.

In some embodiments, the therapeutic radioisotope Z is ¹⁷⁷Lu, ⁶⁷Cu, ⁹⁰Y, ¹⁸⁶Re or ¹⁸⁸Re.

In some preferred embodiments, the therapeutic radioisotope is ¹⁷⁷Lu or ⁶⁷Cu.

In some embodiments, the compound according to Formula (II) is provided in a buffer, wherein the buffer has a pH of about 5.0.

In some embodiments, the process comprises eluting the therapeutic radioisotope onto a strong cation exchange column, and eluting the strong cation exchange column into a compound according to Formula (II).

In some embodiments, the therapeutic radioisotope is added to the compound according to Formula (II) in the presence of one or more antioxidants. In some embodiments, the one or more antioxidants comprise ascorbic acid. In some embodiments, the concentration of the ascorbic acid in the reaction mixture is about 0.01 M or greater.

In some embodiments, the therapeutic radioisotope is added to the compound according to Formula (II) in the presence of glutathione. In some embodiments, the therapeutic radioisotope is added to the compound according to Formula (II) in the presence of both glutathione and ascorbic acid. In some embodiments, the concentration of glutathione in the reaction mixture is about 0.01 M or greater.

According to a seventh aspect, the present disclosure provides a process for preparing a compound according to Formula (I)

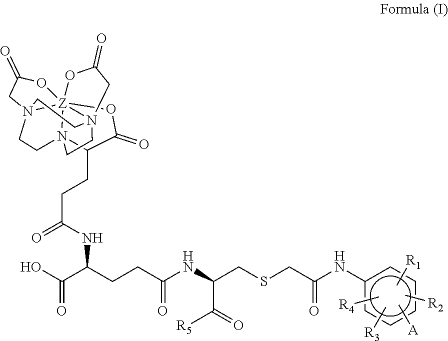

wherein A is —As(OH)₂ or an arsenoxide equivalent group;
each of R₁, R₂, R₃ and R₄ is independently selected from H, X, OH, NH₂, CO, SCN, —CH₂NH, —NHCOCH₃, —NHCOCH₂X or NO, and X is a halogen;
R₅ is —NHCH₂COOH, OH or OR₆, wherein R₆ is a C₁₋₅ straight or branched alkyl group; and Z is a radioisotope,
or a pharmaceutically acceptable salt, ester, prodrug or solvate thereof,
said process comprising adding the radioisotope to a compound according to Formula (II)

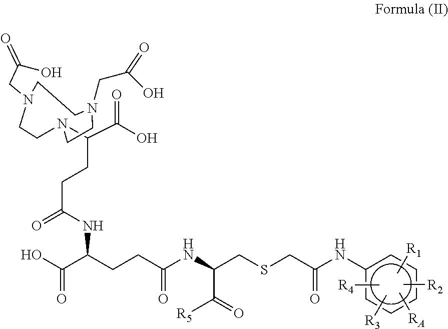

wherein A is —As(OH)₂ or an arsenoxide equivalent group;
each of R₁, R₂, R₃ and R₄ is independently selected from H, X, OH, NH₂, CO, SCN, —CH₂NH, —NHCOCH₃, —NHCOCH₂X or NO, and X is a halogen;
R₅ is —NHCH₂COOH, OH or OR₆, wherein R₆ is a C₁₋₅ straight or branched alkyl group;
or a pharmaceutically acceptable salt, ester, prodrug or solvate thereof, wherein the radioisotope is added to the compound of Formula (II) in the presence of glutathione. In some embodiments, the concentration of glutathione in the reaction mixture is about 0.01 M or greater.

In some embodiments, the radioisotope is added to the compound of Formula (II) in the presence of one or more antioxidants, for example ascorbic acid. In some embodiments, the concentration of the ascorbic acid in the reaction mixture is about 0.01 M or greater.

In particular embodiments, the radioistotope is a therapeutic radioisotope as defined in the first aspect, and/or a radioisotope with a half-life of less than 4 days for example ⁶⁸Ga.

### BRIEF DESCRIPTION OF THE DRAWINGS

Exemplary embodiments of the present disclosure are described herein, by way of non-limiting example only, with reference to the following drawings.

FIG. 1 shows the model of action for the therapeutic radiolabelled compounds disclosed herein.

FIG. 2 shows the HPLC chromatogram of ¹⁷⁵Lu-NODAGA-GSAO labelled for 30 minutes at pH 5.0 at 80° C., (A) without 2,3-dimercapto-1-propanol (DMP) or (B) with pre-incubation with DMP as described in Example 2.

FIG. 3 shows the HPLC chromatogram of ⁶³Cu-NODAGA-GSAO labelled for 30 minutes at pH 5.0 at room temperature, (A) without DMP or (B) with pre-incubation with DMP as described in Example 2.

FIG. 4 shows the HPLC chromatogram of ⁸⁹Y-NODAGA-GSAO labelled for 30 minutes at pH 5.0 at 120° C. as described in Example 2.

FIG. 5 shows the percentage of labelling at different timepoints following formation of the isotope-NODAGA-GSAO complex for A) the ¹⁷⁵Lu-labelled product obtained from incubation for 30 minutes at pH 5.0 at 80° C. and B) the ⁶³Cu-labelled product obtained from incubation for 30 minutes at pH 5.0 at room temperature.

FIG. 6 is a radiometric HPLC chromatogram of the reaction product of Example 4 at the end of synthesis.

FIG. 7 is a radiometric HPLC chromatogram of the reaction product of Example 4 1.5 hours after the end of synthesis.

FIG. 8 is a radiometric HPLC chromatogram of the reaction product of Example 5a at the end of synthesis.

FIG. 9 is a radiometric HPLC chromatogram of the reaction product of Example 5a mixed with 1% DMP in DMSO.

FIG. 10 is a radiometric HPLC chromatogram of the reaction product of Example 5b at the end of synthesis.

FIG. 11 is a radiometric HPLC chromatogram of the reaction product of Example 5b mixed with 1% DMP in DMSO.

FIG. 12 is a radiometric HPLC chromatogram of the reaction product of Example 5c at the end of synthesis.

FIG. 13 is a radiometric HPLC chromatogram of the reaction product of Example 5c at the end of synthesis mixed with 1% DMP in DMSO.

FIG. 14 is a radiometric HPLC chromatogram of the reaction product of Example 5c at 72 hours post-synthesis.

FIG. 15 is a radiometric HPLC chromatogram of the reaction product of Example 5c at 72 hours post-synthesis mixed with 1% DMP in DMSO.

FIG. 16 is a radiometric HPLC chromatogram of the reaction product of Example 5d at the end of synthesis.

FIG. 17 is a radiometric HPLC chromatogram of the reaction product of Example 5d at the end of synthesis mixed with 1% DMP in DMSO.

FIG. 18 is a schematic diagram of a radiolabelling system as used in Example 6.

FIG. 19 is a radiometric HPLC chromatogram of the final product produced in Example 6.

FIG. 20 is a radiometric HPLC chromatogram of the final product produced in Example 6 (the same product as FIG. 19) following reaction with DMP.

FIG. 21 shows the biodistribution of ⁶⁸Ga-NODAGA-GSAO (% ID/g) in healthy male rats at 1 and 2 hours post administration of ⁶⁸Ga-NODAGA-GSAO.

FIG. 22 shows the maximum intensity projection of ⁶⁸Ga-NODAGA-GSAO PET CT scans performed a) 1 hour and b) 2 hours following tracer (⁶⁸Ga-NODAGA-GSAO) administration.

FIG. 23 shows anterior maximum intensity projections of ⁶⁸Ga-NODAGA-GSAO PET at 8 time points following injection in patient 1.

FIG. 24 shows anterior maximum intensity projections of ⁶⁸Ga-NODAGA-GSAO PET at 8 time points following injection in patient 2.

FIG. 25 shows anterior maximum intensity projections of ⁶⁸Ga-NODAGA-GSAO PET at 8 time points following injection in patient 3.

FIG. 26 shows anterior maximum intensity projections of ⁶⁸Ga-NODAGA-GSAO PET at 8 time points following injection in patient 4.

FIG. 27 shows biodistribution of ⁶⁸Ga-NODAGA-GSAO in normal organs of patient 1 over time.

FIG. 28 shows biodistribution of ⁶⁸Ga NODAGA GSAO in selected normal tissues and tumour for patient 1.

FIG. 29 shows biodistribution of ⁶⁸Ga NODAGA GSAO in selected normal tissues and tumour for patient 2.

FIG. 30 shows biodistribution of ⁶⁸Ga NODAGA GSAO in selected normal tissues and tumour for patient 3.

FIG. 31 shows biodistribution of ⁶⁸Ga NODAGA GSAO in selected normal tissues and tumour for patient 4.

FIG. 32 shows the biodistribution in selected normal tissues (mean SUV±SD) of ⁶⁸GaNODAGA GSAO in subjects 1-4.

FIG. 33 shows blood pool activity and uptake of ⁶⁸Ga NODAGA GSAO into tumour deposits in subjects 1-4.

FIG. 34 shows anterior maximum projection intensity images of FDG-PET (FIG. 34A) performed 60 min after administration of 256 MBq of FDG (fluorodeoxyglucose), and CDI-PET (FIG. 34B) performed 60 min after administration of 205 MBq of CDI (⁶⁸Ga NODAGA GSAO) in patient 3. The tumours were surgically excised, fixed and adjacent sections stained for apoptotic cells (FIG. 34C, brown TUNEL stain, a and b) or for morphology by haematoxylin and eosin (FIG. 34C, c and d).

### DETAILED DESCRIPTION OF THE INVENTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

Throughout this specification, unless the context requires otherwise, the word “comprise”, or variations such as “comprises” or“comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the terms “a” and “an” refer to one or to more than one (i.e. to at least one) of the grammatical object of the article. By way of example, “an element” means one element or more than one element.

In the context of this specification, the term “about” is understood to refer to a range of numbers that a person of skill in the art would consider equivalent to the recited value in the context of achieving the same function or result.

In the context of this specification, reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein, the term “and/or” means “and” or “or” or both.

The term “subject” as used herein refers to any mammal, including, but not limited to, livestock and other farm animals (such as cattle, goats, sheep, horses, pigs and chickens), performance animals (such as racehorses), companion animals (such as cats and dogs), laboratory test animals and humans. Typically the subject is a human.

As used herein the terms “treating”, “treatment”, “treating”, “reduce”, “reducing”, “prevent” “preventing” and “prevention” and the like refer to any and all applications which remedy, or otherwise hinder, retard, or reverse the progression of, an infection or disease or at least one symptom of an infection or disease, including reducing the severity of an infection or disease. Thus, the terms “treat”, “treating”, “treatment”, do not necessarily imply that a subject is treated until complete elimination of the infection or recovery from a disease. Similarly, the terms “prevent”, “preventing”, “prevention” and the like refer to any and all applications that prevent the establishment of an infection or disease or otherwise delay the onset of an infection or disease.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

As used herein the terms “effective amount” and “effective dose” include within their meaning a non-toxic but sufficient amount or dose of a compound to provide the desired effect. The exact amount or dose required will vary from subject to subject depending on factors such as the species being treated, the age and general condition of the subject, the severity of the condition being treated, the particular compound being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

In the context of the present specification, the term “arsenoxide” refers to the group —As═O. The groups written —As═O and —As(OH)₂ are to be considered synonymous.

As used herein, the term “arsenoxide equivalent” refers to any dithiol reactive species that shows essentially the same affinity towards dithiols as —As═O or As(OH)₂, and the term includes, for example, groups comprising a transition element, and any trivalent arsenical that is either hydrolysed to —As═O or —As(OH)₂ when dissolved in aqueous medium (such as cell culture buffers and the fluids contained in the organism being treated). Typically, arsenoxide equivalent includes dithiol reactive entities, such as As, Ge, Sn and Sb species. Arsenoxide equivalents are expected to exhibit identical or substantially identical activity to that of the corresponding arsenoxide.

The term “bifunctional chelator” refers to a chemical moiety which comprises a chelating moiety capable of binding a metal or other ion, for example a radionuclide, as well as a chemically reactive functional group for attachment to a further chemical entity. In the context of the present application, the term “bifunctional chelator” refers to both the relevant chemical compound before chelation with a metal or other ion and/or before reaction at the reactive functional group, as well as once chelated to a metal or other ion and/or attached to a further chemical entity by way of the reactive functional group, the relevant definition being readily apparent from context. When not chelating a metal or other ion, a bifunctional chelator is suitable for chelating a metal or other ion.

The terms “C₁-C₅-alkyl”, or the like, as used herein, refer to saturated, straight- or branched-chain hydrocarbon radicals containing between one and three, one and six or one and twelve carbon atoms, respectively. Examples of C₁-C₅-alkyl radicals include but are not limited to methyl, ethyl, propyl, isopropyl, n-butyl, sec-butyl, isobutyl, tert-butyl, pentyl, isopentyl and neopentyl.

By “pharmaceutically acceptable salt” it is meant those salts which, within the scope of sound medical judgement, are suitable for use in contact with the tissues of humans and lower animals without undue toxicity, irritation, allergic response and the like, and are commensurate with a reasonable benefit/risk ratio. Reference to a compound herein shall be understood to include its pharmaceutically acceptable salts unless specified otherwise or otherwise understood from context.

Provided herein are compounds according to Formula (X)

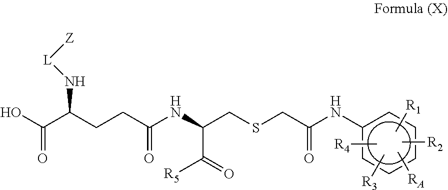

wherein A is —As(OH)₂ or an arsenoxide equivalent group; each of R₁, R₂, R₃ and R₄ is independently selected from H, X, OH, NH₂, CO, SCN, —CH₂NH, —NHCOCH₃, —NHCOCH₂X or NO, and X is a halogen; R₅ is —NHCH₂COOH, OH or OR₆, wherein R₆ is a C₁₋₅ straight or branched alkyl group; Z is a therapeutic radioisotope; and L is a bifunctional chelator chelating Z; or a pharmaceutically acceptable salt, ester, prodrug or solvate thereof.

In some embodiments, the compound according to Formula (X) is a compound according to Formula (Xa), or pharmaceutically acceptable salt, ester, prodrug or solvate thereof.

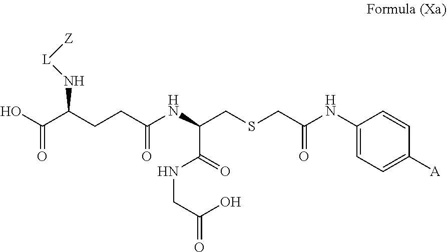

A “therapeutic radioisotope” is, in the context of the present disclosure, understood to refer to any radioisotope which has a therapeutic effect, in particular a therapeutic effect in promoting cell death, for example for the treatment of a neoplastic condition, such as a tumour or cancer. A “neoplastic condition” is understood to refer to a condition characterised by abnormally high levels of cell proliferation. Such promotion of cell death is to a therapeutically useful degree. Typically, the therapeutic isotope is an alpha or beta emitter. In some embodiments, the therapeutic isotope is an alpha emitter, for example ²²⁵Ac, ²¹¹At, ²¹³Bi and/or ²²³Ra. Any suitable therapeutic isotope may be selected, and in particular a therapeutic isotope having the appropriate radius of energy delivery (i.e. cell kill radius) may be selected for particular in vivo biology and the particular desired therapeutic use, for example depending on tumour size/radius and/or the pattern of dispersion of dead and dying cells within the tumour. In some embodiments, Z is selected from ²²⁵Ac ²¹¹At, ²¹³Bi, ²²³Ra.¹⁷⁷Lu, ⁶⁷Cu, ⁶⁴Cu, ⁹⁰Y, ¹⁸⁶Re and ¹⁸⁸Re, for example selected from ¹⁷⁷Lu, ⁶⁷Cu, ⁶⁴Cu, ⁹⁰Y, ¹⁸⁶Re and ¹⁸⁸Re. In some embodiments, Z is selected from ¹⁷⁷Lu, ⁶⁷Cu, ⁹⁰Y, ¹⁸⁶Re and ¹⁸⁸Re. In some preferred embodiments, Z is ¹⁷⁷Lu, ⁶⁷Cu, ⁶⁴Cu or ⁹⁰Y, for example ¹⁷⁷Lu, ⁶⁷Cu or ⁹⁰Y. In particularly preferred embodiments, Z is ¹⁷⁷Lu or ⁶⁷Cu. In particular embodiments wherein Z is a Re isotope, such as ¹⁸⁸Re or ¹⁸⁶Re, or other therapeutic isotopes as necessary, Z may refer to the therapeutic isotope in any suitable form for incorporation into the compounds of the present disclosure, for example as a tricarbonyl, for example rhenium tricarbonyl. In preferred embodiments, L is a bifunctional chelator known to chelate a therapeutic radioisotope, for example ¹⁷⁷Lu, ⁶⁷Cu, or ⁹⁰Y, with a high affinity. In some preferred embodiments, the therapeutic isotope also functions as a diagnostic isotope, i.e. has a diagnostic emission to enable imaging of the compound, in particular where the therapy has been delivered and or/how much therapeutic compound has been delivered to the desired location, and/or calculation of radiation dose to tumour and normal tissue to determine probability of tumour kill and also normal tissue toxicity. For example, the therapeutic isotope may be positron emitting and be imaged by positron emission tomography. In some embodiments, imaging may be carried out by single photon imaging (SPECT). In some embodiments, nuclear medicine (‘gamma camera’) may be used to image the radioisotope. The particular type of imaging suited to a given isotope and application will be readily apparent to a skilled person.

In some embodiments. Z is not ⁶⁴Cu.

The present disclosure provides a compound according to Formula (I)

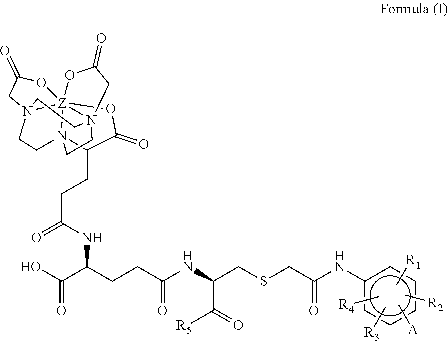

wherein A is —As(OH)₂ or an arsenoxide equivalent group; each of R₁, R₂, R₃ and R₄ is independently selected from H, X, OH, NH₂, CO, SCN, —CH₂NH, —NHCOCH₃, —NHCOCH₂X or NO, and X is a halogen; R₅ is —NHCH₂COOH, OH or OR₆, wherein R₆ is a C₁₋₅ straight or branched alkyl group; and Z is a therapeutic radioisotope, or a pharmaceutically acceptable salt, ester, prodrug or solvate thereof. In some preferred embodiments, each of R₁, R₂, R₃ and R₄ are H. In some preferred embodiments, R₅ is —NHCH₂COOH. In particular preferred embodiments, the compound is a compound according to Formula (Ia):

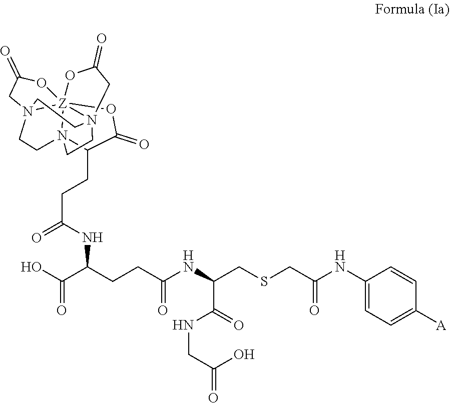

or a pharmaceutically acceptable salt, ester, prodrug or solvate thereof. In preferred embodiments, A is an arsenoxide group As(OH)₂.

In compounds suitable for use in the present invention, the arsenoxide group (—As(OH)₂) can typically be replaced by an arsenoxide equivalent.

Such compounds are based on 4-(N—(S-glutathionylacetyl)amino)phenylarsenous acid (GSAO) which has been radiolabelled with a radioisotope using a bifunctional chelator. In particularly preferred embodiments, the bifunctional chelator is 2,2′-(7-(1-carboxy-4-((2,5-dioxopyrrolidin-1-yl)oxy)-4-oxobutyl)-1,4,7-triazonane-1,4-diyl)diacetic acid (NODAGA), as shown in Formula (I) and Formula (Ia).

GSAO undergoes specific uptake into dead and dying cells. Without wishing to be bound by theory, it is thought that GSAO is retained in the cytosol of dying and dead cells via the formation of covalent bonds between the As(III) ion and the thiol groups of proximal cysteine residues. GSAO is a trivalent As(III) peptide, which has been found to activate the mitochondrial permeability transition pore. GSAO is toxic to proliferating cells and inhibits angiogenesis in vivo (Don A S, Kisker O, Dilda P et al (2003) A peptide trivalent arsenical inhibits tumor angiogenesis by perturbing mitochondrial function in angiogenic endothelial cells. Cancer Cell 3:497-509), but is nontoxic to quiescent endothelial cells in vitro. Conjugation of the γ-glutamyl residue of GSAO with therapeutic radioisotopes, as in the present invention, results in loss of its anti-angiogenic effect and the ability to target dying cells. When the plasma membrane integrity has been compromised the GSAO conjugate enters and binds to intra-cellular proteins, predominantly 90 kDa heat-shock proteins (Hsp90) (Park D, Don A S, Massamiri T et al (2011) Non-invasive imaging of cell death using an Hsp90 ligand. J Am Chem Soc 133:2832-2835); this protein is highly abundant in the cytosol, is only accessible when cell membrane integrity is compromised during cell death and is up-regulated in many malignancies (Hahn J S. The Hsp90 chaperone machinery: from structure to drug development. BMB Rep. 2009; 42(10):623-30). The As(III) motif of GSAO cross-links the unpaired thiols of Cys597 and Cys598 of Hsp90 forming a stable cyclic dithioarsinite which is effectively irreversible in the cell cytosl. The radiolabelled compounds of embodiments of the present disclosure do not target a transitory cell death process, recognise both apoptotic and necrotic forms of cell death, and the cellular target is abundant and the binding irreversible. These characteristics are especially advantageous for providing a targeted therapeutic agent targeting areas of cell death.

In Examples 4-8 of the present disclosure, biodistribution of compounds analogous to those of embodiments of the present invention, albeit radiolabelled with ⁶⁸Ga rather than a therapeutic isotope, have also been shown to be readily manufactured and have favourable human biodistribution characteristics, with high uptake in tumours and low uptake in normal tissues, and with no observed adverse events. Low uptake in normal tissues minimises side effects of treatment with compounds according to embodiments of the present disclosure.

When in situ at an area of high cell death such as a tumour, radiation emitted by the therapeutic radioisotope is of a range that affects multiple surrounding cells. Compounds according to the present disclosure labelled with therapeutic radioisotopes label dying and dead cells such as tumour cells with high specificity and sensitivity and are thus useful for providing therapeutic radioisotopes specifically to areas of high cell death, such as tumours. In particular, radiolabelled conjugates as described herein can be used in treating conditions associated with high levels of cell death, for example neoplastic disorders, for example tumours or for example cancer. Since the compounds of the present disclosure are targeted to areas of high cell death and cell turnover, such as tumours, they may be advantageously used to selectively enhance tumour cell death by delivering therapeutic isotopes to tumours, such that therapeutic radiation is consequently delivered to viable tumour cells adjacent to dying/dead cells; these adjacent cells may be relatively resistant to other treatment given that they are not already committed to cell death. Induction of death in adjacent cells by the radioisotope may then also further promote binding of the radiolabelled compound of the present disclosure, consequently causing further cell death in adjacent cells. This may create a positive-feedback mechanism for treating conditions such as tumours. Compounds according to the present disclosure also find use in “amplifying” approaches to therapy, wherein an initiator event causing cell death, such as a therapy such as radiotherapy, chemotherapy, immunotherapy or targeted therapy, is carried out, which increases the number of dying cells in a target area, and a radiolabelled compound of the present disclosure is also administered, which binds to said dying cells (whether before, after or concurrently with the initiator therapy). The binding of the radiolabelled compound of the present disclosure to the dying cells causes further cell death in adjacent cells, as discussed above, thus amplifying the effects of the initiator therapy, such as radiotherapy, chemotherapy, immunotherapy or targeted therapy.

For example, compounds of the present disclosure may be administered in combination (including at different times) with another therapy, such as a further radiopharmaceutical, such as a further targeted radiopharmaceutical, in order to reduce the dose required of the other therapy by virtue of further cell death also being induced by the compound of the present disclosure. Compounds of the present disclosure may also enhance the efficacy of a therapy by extending its efficacy, for example by inducing cell death in cells which are not targeted by another targeted therapy; for example, in circumstances wherein a subject is suffering from two different subsets of cancer which express different markers and a targeted therapy is only targeted to one such subset, compounds of the present disclosure may be used to induce cell death in those cells not targeted by the alternative targeted therapy. As such, the present disclosure provides methods for reducing the dose of a therapy required to effectively treat a condition and/or enhancing the effectiveness of a therapy, for example of a radiopharmaceutical such as a targeted radiopharmaceutical, comprising administering the therapy in combination with a compound according to the present disclosure. Such administration in combination may include administering the therapy and the compound of the present disclosure at different times or concurrently.

In such treatments, the target is the area of cell death, such as the tumour itself, rather than an area adjacent to a tumour, providing a treatment having high specificity. This mechanism provides a particularly efficient and targeted means of treating conditions such as tumours. Radiolabelled compounds according to embodiments of the present disclosure may further advantageously target all sites of disease whilst sparing normal tissues, which is particularly helpful in the treatment of non-localised cancers. The radiolabelled conjugates of the present disclosure may, in some embodiments, advantageously provide a valuable pan-tumour treatment, since dying/dead tumour cells are present in all solid tumours.

As noted above, compounds analogous to those of the present disclosure, but using ⁶⁸Ga rather than a therapeutic isotope, are shown by Examples 6-8 of the present application to have very low levels of activity in all organs except the urinary tract that is the route of excretion, with the dose limiting organ being the urinary bladder. This is in common with the radionuclides used for treatment of neuroendocrine tumours and there are established protocols for management of this. Importantly, targeting of the compound to cell death in healthy tissues such as bone marrow, lymph nodes and the gastrointestinal tract is minimal, if at all. Dying cells in normal tissues are cleared rapidly by macrophages, unlike dying/dead cells in tumours that persist for days to weeks, which may be why targeting to cell death in healthy tissues is minimal. High uptake of the compound within tumours with high basal cell death is also demonstrated by the Examples of the present disclosure (Example 8). This greatly minimises potential side-effects of the therapeutic compounds.

According to some preferred embodiments, Z may be, for example, ¹⁷⁷Lu, ⁶⁷Cu, ⁶⁴Cu, ⁹⁰Y, ¹⁸⁶Re or ¹⁸⁸Re. In some embodiments, Z may be, for example, ¹⁷⁷Lu, ⁶⁷Cu, ⁹⁰Y, ¹⁸⁶Re or ¹⁸⁸Re. In some preferred embodiments, Z is ¹⁷⁷Lu, ⁶⁷Cu, ⁶⁴Cu or ⁹⁰Y. In some embodiments, Z is ¹⁷⁷Lu, ⁶⁷Cu or 9° Y, or, in some embodiments, Z is ¹⁷⁷Lu, ⁶⁷Cu or ⁶⁴Cu. In particularly preferred embodiments, Z is ¹⁷⁷Lu or ⁶⁷Cu. ¹⁷⁷Lu is a decaying radioactive atom that emits beta particles, which are negatively charged electrons, with a maximum energy of 497 keV that travel ˜1,800 μm in biological tissue. Tumour cells have diameters of 10-20 μm, so ¹⁷⁷Lu beta particles can travel the width of several tumour cells. Labelling of dying and dead tumour cells with a ¹⁷⁷Lu labelled compound according to embodiments of the present disclosure therefore delivers therapeutic radiation to adjacent, viable tumour cells. ¹⁷⁷Lu has a half-life of 6.7 d, which is well suited for a therapeutic isotope. ⁶⁷Cu has been proven to be clinically useful for the treatment of cancer. ⁹⁰Y is used in a wide range of applications in radiation therapy, including as treatment strategy for certain forms of cancer. In some embodiments, Z is not ⁶⁴Cu.

In particularly preferred embodiments, the compound according to Formula I is ¹⁷⁷Lu-NODAGA-GSAO (i.e. the compound of Formula I wherein Z is ¹⁷⁷Lu, R₁-R₄ are H, R₅ is —NHCH₂COOH, and A is As(OH)₂) or ⁶⁷Cu-NODAGA-GSAO (i.e. the compound of Formula I wherein Z is ⁶⁷Cu, R₁-R₄ are H, R₅ is —NHCH₂COOH, and A is As(OH)₂). Such embodiments provide the advantages of being readily synthesised and providing radioisotopes useful for treatment of cancers in a targeted manner.

According to a further aspect, the present disclosure also provides compounds according to Formula (Y)

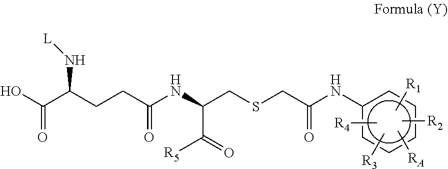

wherein A is —As(OH)₂ or an arsenoxide equivalent group; each of R₁, R₂, R₃ and R₄ is independently selected from H, X, OH, NH₂, CO, SCN, —CH₂NH, —NHCOCH₃, —NHCOCH₂X or NO, and X is a halogen; R₅ is —NHCH₂COOH, OH or OR₆, wherein R₆ is a C₁₋₅ straight or branched alkyl group; and L is a bifunctional chelator; or a pharmaceutically acceptable salt, ester, prodrug or solvate thereof or derivative thereof.

In some preferred embodiments, the compound according to Formula (Y) is a compound according to Formula (Ya)

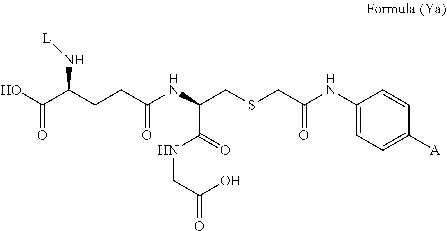

The present disclosure provides a compound according to Formula (Y) which is a compound according to Formula II

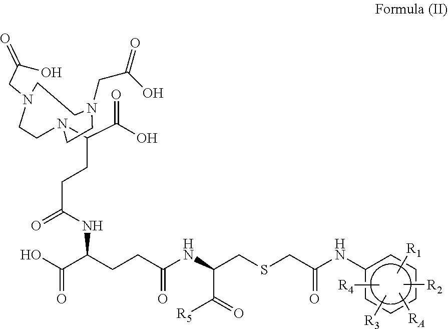

wherein A is —As(OH)₂ or an arsenoxide equivalent group; each of R₁, R₂, R₃ and R₄ is independently selected from H, X, OH, NH₂, CO, SCN, —CH₂NH, —NHCOCH₃, —NHCOCH₂X or NO, and X is a halogen; R₅ is —NHCH₂COOH, OH or OR₆, wherein R₆ is a C₁₋₅ straight or branched alkyl group; or a pharmaceutically acceptable salt, ester, prodrug or solvate thereof.

Compounds of Formula (Y) are useful in the synthesis of Formula (X). In particular, compounds according to Formula (II) are useful in the synthesis of compounds according to Formula I, by radiolabelling of the NODAGA group. Such a synthesis is represented schematically below in Scheme 1, exemplified by NODAGA-GSAO as the starting material and ¹⁷⁷Lu as the radioisotope.

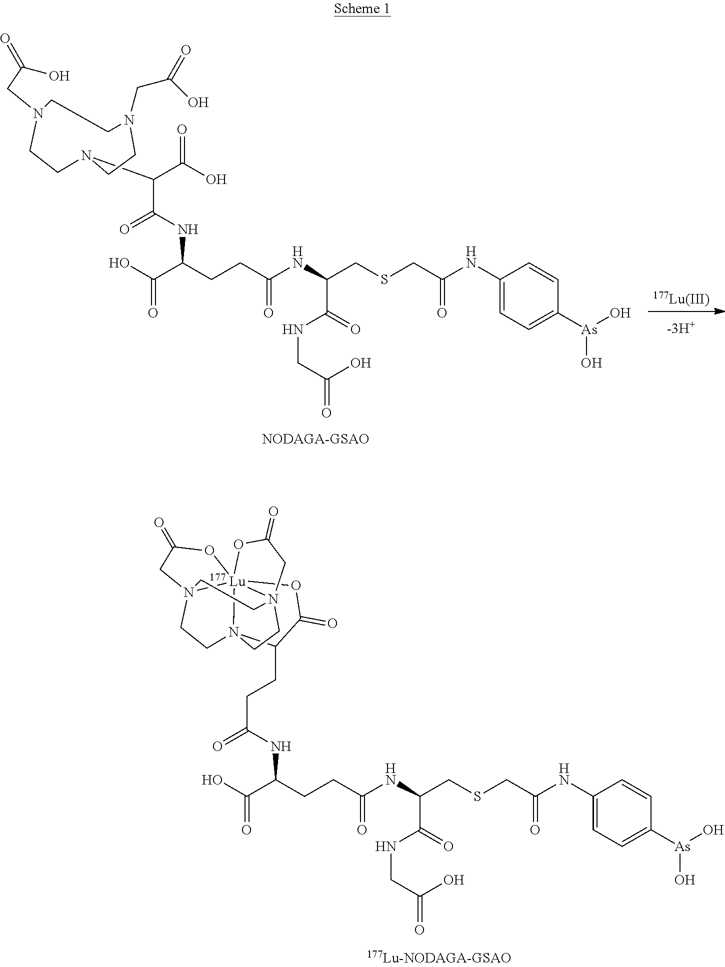

In preferred embodiment, each of R₁, R₂, R₃ and R₄ are H. In further preferred embodiments, R₅ is —NHCH₂COOH. In particularly preferred embodiments, the compound is a compound according to Formula (IIa)

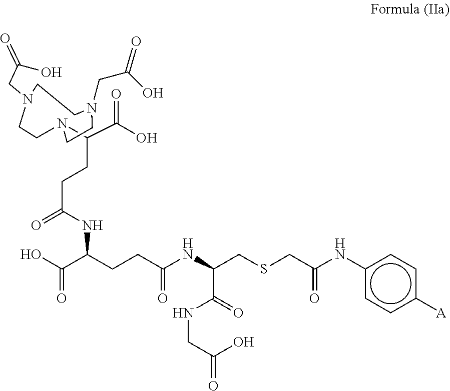

wherein A is as defined for Formula (II); or a pharmaceutically acceptable salt, ester, prodrug or solvate thereof.

In preferred embodiments, A is an arsenoxide group As(OH)₂.

In compounds suitable for use in the present invention, the arsenoxide group (—As(OH)₂) can typically be replaced by an arsenoxide equivalent.

The present disclosure provides a process for preparing a compound according to Formula (I) comprising mixing a therapeutic radioisotope with a compound according to Formula (II), wherein the compound of Formula (I) or Formula (II) may be any of those described above. In some embodiments, the mixing takes place at room temperature, i.e. without heating, for example in some embodiments wherein Z is 67Cu. In some embodiments, the mixing takes place with heating, for example in some embodiments wherein Z is 177Lu. In such embodiments, the heating may be to a temperature of, for example, at least about 60° C., for example from about 60° C. to about 80° C., for example about 80° C., for example in embodiments wherein Z is 177Lu or, in some embodiments, the heating may be to a temperature of, for example, at least about 80° C., for example from about 80° C. to about 150° C., for example about 120° C., for example in embodiments wherein Z is 9° Y. In some embodiments, the mixing occurs at a pH of at least about 5.0, for example about 5.0, for example in embodiments wherein Z is ¹⁷⁷Lu. In some particular preferred embodiments wherein Z is ¹⁷⁷Lu, the mixing occurs at a temperature from about 60 to about 80° C., at a pH of at least about 5.0, for example about 5.0, optionally for a time period of about at least 20 minutes, for example about 30 minutes. Desired pH levels may be achieved by use of any suitable buffer, for example sodium acetate buffer.

The present disclosure provides a process for preparing a compound according to Formula (I), comprising adding therapeutic radioisotope Z to a compound according to Formula (II). The compound according to Formula (II) may, according to some embodiments, be optionally mixed with a buffer, wherein the buffer may have a pH of, for example, at least about 5.0, for example about 5.0. In some embodiments, the mixing is carried out at room temperature, i.e. without heating, and in some alternative embodiments, the mixing is carried out with heating, as described above. In some embodiments, the process comprises eluting the therapeutic radioisotope onto a strong cation exchange column, and eluting the strong cation exchange column into a compound according to Formula (II). In some embodiments, the compounds according to Formula (I) and Formula (II) are compounds according to Formula (Ia) and Formula (IIa) respectively. The present disclosure further provides a process for preparing a compound according to Formula (X), comprising mixing a therapeutic radioisotope with a compound according to Formula (Y), wherein the compound of Formula (X) or Formula (Y) may be any of those described above. In some embodiments, the mixing is carried out at room temperature, i.e. without heating, and in some alternative embodiments, the mixing is carried out with heating, as described above. The present disclosure provides a process for preparing a compound according to Formula (X) wherein Z is ¹⁷⁷Lu, ⁶⁴Cu or ⁶⁷Cu, such as ¹⁷⁷Lu or ⁶⁷Cu comprising adding ¹⁷⁷Lu or ⁶⁷Cu to a compound according to Formula (Y), optionally mixed with a buffer, wherein the buffer has, in some embodiments, a pH of at least about 5.0, for example about 5.0. In some embodiments, the mixing is carried out at room temperature, i.e. without heating, for example in some embodiments wherein Z is ⁶⁷Cu or 64Cu, such as ⁶⁷Cu. In some alternative embodiments, the mixing is carried out with heating, as described above, for example in some embodiments wherein Z is ¹⁷⁷Lu.

In some embodiments of the processes described above, the therapeutic radioisotope is added to the compound according to Formula (II) (or Formula (IIa) or Formula (Y) as described herein) in the presence of one or more antioxidants. In particular embodiments, the one or more antioxidants comprise ascorbic acid. In some embodiments of the processes described above, the therapeutic radioisotope is added to the compound according to Formula (II) (or Formula (IIa) or Formula (Y) as described herein) in the presence of one or more protectants against radiolysis. In some embodiments of the processes described above, the therapeutic radioisotope is added to the compound according to Formula (II) (or Formula (IIa) or Formula (Y) as described herein) in the presence of glutathione. Without wishing to be bound by theory, it is thought that glutathione may function both as an antioxidant and as a protectant in reducing radiolytic breakdown of NODAGA-GSAO during synthesis which produces oxidised NODAGA-GSAO. “Added to” indicates that the therapeutic radioisotope is reacted with the compound according to Formula (II) in the presence of the one recited components, regardless of in which order the components are added to a reaction mixture. As demonstrated in Examples 4 and 5 below, the presence of an antioxidant such as ascorbic acid, and/or the presence of glutathione, and in particular the presence of both ascorbic acid and glutathione, especially in high concentrations, reduced radiolytic breakdown of NODAGA-GSAO during synthesis which produces oxidised NODAGA-GSAO. Accordingly, in preferred embodiments, the therapeutic radioisotope is added to the compound according to Formula (II) (or Formula (IIa) or Formula (Y) as described herein) in the presence of one or more antioxidants and/or one or more protectants against radiolysis, for example in the presence of glutathione, for example in the presence of ascorbic acid and glutathione.

In particular embodiments, the one or more antioxidants and/or the one or more protectants, such as ascorbic acid and/or glutathione, may each be present in the reaction mixture in a concentration of about 0.0075 or greater, for example about 0.01 M or greater, for example about 0.0125 or greater, for example about 0.015 or greater, for example about 0.0175 or greater, for example about 0.02 or greater. In some embodiments, the one or more antioxidants and/or the one or more protectants, such as ascorbic acid and/or glutathione, may each be present in the reaction mixture in a concentration of up to about 0.1 M. Where multiple antioxidants and/or protectants are present, such concentrations may, according to some embodiments, relate to each of the separate antioxidants and/or protectants, for example to each of ascorbic acid and/or glutathione independently. The concentration of “in the reaction mixture” refers to the concentration in which the relevant component is present when the therapeutic radioisotope is reacted with the compound according to Formula (II) (or Formula (IIa) or Formula (Y) as described herein).

Such processes may be used to prepare radiolabelled compounds as described herein, except wherein Z is a radioisotope which is not limited to a therapeutic radioisotope. Z may be a therapeutic radioisotope as described herein or Z may be an alternative radioisotope. In some embodiments, Z is a radioisotope with a half-life of less than 4 days, for example less than 1 day, for example less than 4 hours, for example less than 2 hours. Z may, in some embodiments, be a radioisotope suitable for use as an imaging agent, such as in positron emission tomography. In some embodiments, Z is ⁶⁸Ga. Such embodiments may find use in preparing compounds useful in imaging cell death. Z may be as described in PCT application no. PCT/AU2020/050359 (published as WO2020206503).

The present disclosure further provides pharmaceutical compositions and/or therapeutic formulations, that is, compounds of the present disclosure present together with a pharmaceutical acceptable carrier, excipient, diluent and/or vehicle.

For medical use, salts of the compounds according to the present disclosure may be used and they include pharmaceutically acceptable salts, although other salts may be used in the preparation of the compound or of the pharmaceutically acceptable salt thereof. By pharmaceutical acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio.

Pharmaceutically acceptable salts are well known in the art.

For instance, suitable pharmaceutically acceptable salts of the compounds of the present disclosure may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the invention. Suitable pharmaceutically acceptable salts of the compounds of the present disclosure therefore include acid addition salts.

For example, S. M Berge et al describe pharmaceutically acceptable salts in detail in J Pharmaceutical Sciences, 1977, 66: 1-19. The salts can be prepared in situ during the final isolation and purification of the compounds of the present disclosure, or separately by reacting the free base function with a suitable organic acid. Representative acid addition salts include acetate, adipate, alginate, ascorbate, asparate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleat, malonate, methanesulfonate, 2-naphthalenesulfonate, nicotinate, nitrate, oleate, oxalate, palmitat, pamoate, pectinate, persulfate, 3-phenylpropionate, phosphate, picrate, pivalate, propionate, stearate, succinate, sulfate, tartrate, thiocyanate, toluenesulfonate, undecanoate, valerat salts, and the like. Representative alkali or alkaline earth metal salts include sodium, lithium potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The present disclosure also provides prodrugs. Typically, prodrugs will be functional derivatives of the compounds of the present disclosure which are readily converted in vivo to the required (active) compounds of the present disclosure, such as imaging, therapeutic and/or diagnostic agents.

Typical procedures for the selection and preparation of prodrugs are known to those of skill in the art and are described, for instance, in H. Bundgaard (Ed), Design of Prodrugs, Elsevier, 1985.

Intermediates and final products can be worked up and/or purified according to standard methods, e.g., using chromatographic methods, distribution methods, (re-) crystallization, and the like. The compounds, including their salts, may also be obtained in the form of solvates, in particular hydrates. In the context of the invention, solvates refer to those forms of the compounds according to the present disclosure which, in the solid or liquid state, form a complex by coordination with solvent molecules. Hydrates are a specific form of the solvates in which the coordination is with water. Crystals of the present compounds may, for example, include the solvent used for crystallization. Different crystalline forms may be present.

The present disclosure also relates to those forms of the process of preparing compounds according to the present disclosure in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

Single or multiple administrations of the compounds or pharmaceutical compositions can be carried out with dose levels and patterns being selected by the treating physician. Regardless, the compounds or pharmaceutical compositions of the present disclosure should provide a quantity of the compound sufficient to effectively treat the patient.

One skilled in the art would be able, by routine experimentation, to determine an effective, non-toxic amount of the compounds or pharmaceutical compositions used in the invention which would be required to treat or prevent the disorders and diseases disclosed herein.

A compound of the present disclosure may be administered in a dose of, for example, up to 800 μg. In some particular embodiments, a compound of the present disclosure may be administered in a dose of, for example, up to 700 μg, for example up to 600 μg, for example up to 500 μg, for example up to 400 μg, for example up to 300 μg, for example up to 250 μg, for example up to 200 μg, for example up to 150 μg, for example up to 100 μg, for example up to 50 μg. In some preferred embodiments, a compound of the present disclosure is administered in a dose of up to 200 μg. In some embodiments, the compound of the present disclosure is administered in a dose of less than 50 μg, for example 10 to 50 μg.

Whilst the compounds of the present disclosure may be administered alone, it is generally preferable that the compound be administered as a pharmaceutical composition/formulation. In general pharmaceutical formulations of the compounds of the present disclosure may be prepared according to methods which are known to those of ordinary skill in the art and accordingly may include a pharmaceutically acceptable carrier, excipient, diluent, vehicle and/or adjuvant.

The carriers, excipients, diluents, vehicles and adjuvants must be “acceptable” in terms of being compatible with the other ingredients of the formulation, and not deleterious to the recipient thereof.

In some embodiments, pharmaceutical compositions of the present disclosure comprise a compound according to the present disclosure, as well as one or more further components selected from ascorbic acid, sodium, phosphate, acetate and chloride. In some embodiments, the pharmaceutical compositions comprises all such components. In a preferred form the pharmaceutical composition of a compound of the present disclosure comprises an effective amount of a compound according to the present disclosure, together with the pharmaceutically acceptable carriers, diluents and/or adjuvants as shown in Example 5 for an analogous composition comprising ⁶⁸Ga-NODAGA-GSAO instead of a therapeutic isotope compound as disclosed herein.

The pharmaceutical compositions of the present disclosure may be administered by standard routes.

In particularly preferred embodiments, the compound or pharmaceutical composition of the present disclosure is administered intravenously. For administration as an injectable solution or suspension, non-toxic parenterally acceptable diluents or carriers can include, Ringer's solution, isotonic saline, phosphate buffered saline, ethanol and 1, 2-propylene glycol.

The present disclosure provides compounds and compositions according to the present disclosure for use in the treatment of neoplastic conditions, including tumours and cancers, for example solid tumours. The cancer may include cancers which do not necessarily comprise solid or discrete tumours, for example leukaemia or lymphoma. Said treatment is by delivery of a therapeutic radioisotope to an area of cell death and, in response to delivery of the therapeutic isotope, induction/enhancement of cell death in surrounding cells. When administered intravenously, the compounds of the present disclosure will target dying cells present in high levels, such as in tumours (which have high rates of cell death and turnover); as a consequence, radiation from the therapeutic radioisotope will be delivered to adjacent, viable cells, causing death of surrounding tumour cells. Such cell death induced by compounds of the present disclosure may cause further binding of compound of the present disclosure, thus causing further cell death in a positive-feedback mechanism; compounds of the present disclosure may be administered to a subject multiple times (i.e. in multiple cycles), to provide increased cell death across the multiple cycles, for example with each administration cycle.

The present disclosure further provides methods of treating the above-mentioned conditions comprising administration of a therapeutically effective amount of a compound described herein to a subject. The present disclosure further provides use of the compounds described herein in such methods, and use in the manufacture of medicaments for the treatment of such conditions. Said treatments may be by way of the compounds of the present disclosure comprising a therapeutic isotope inducing cell death, in particular in cells surrounding dying cells which the compounds selectively label.

Use of the compounds of the present disclosure and methods of treatment provided herein, for example of the conditions described above, include administration of an effective amount of a compound or pharmaceutical composition described herein to a subject.

In some embodiments, such methods comprise administering an effective amount of a compound or a pharmaceutical composition of the present disclosure in two or more cycles, wherein efficacy of the administration against the neoplastic condition increases across the two or more cycles. An increase in efficacy of the administration against the neoplastic condition across the two or more cycles may include overall increases in efficacy from the first to a later cycle, even if the efficacy in each individual cycle is not greater than the immediate previous cycle. In some preferred embodiments, the efficacy of the administration against the neoplastic condition increases with each of the two or more cycles, i.e. the efficacy of each administration is greater than in the immediately previous cycle. “Cycles” will be understood to refer to separate, repeated administrations, which may or may not be interspersed by other steps, such as administration of other therapies. An increase in efficacy of the administration against the neoplastic condition across the two or more cycles arises due to the positive feedback mechanism associated with the compounds of the present disclosure discussed above; compounds may exhibit a self-amplifying effect, where cell death caused by compounds of the present disclosure in turn attracts more compound, which in turn induces more cell death. As such, subsequent cycles of administration of a compound of the present disclosure may have increased efficacy against the neoplastic condition by virtue of increased uptake of the compound in dying cells, due to increased levels of cell death caused by previous administration(s). “Increased efficacy” may be understood as higher levels of cell death in the target area (such as a tumour) caused by administration of the compound for a given amount of compound, relative to previous administration(s).

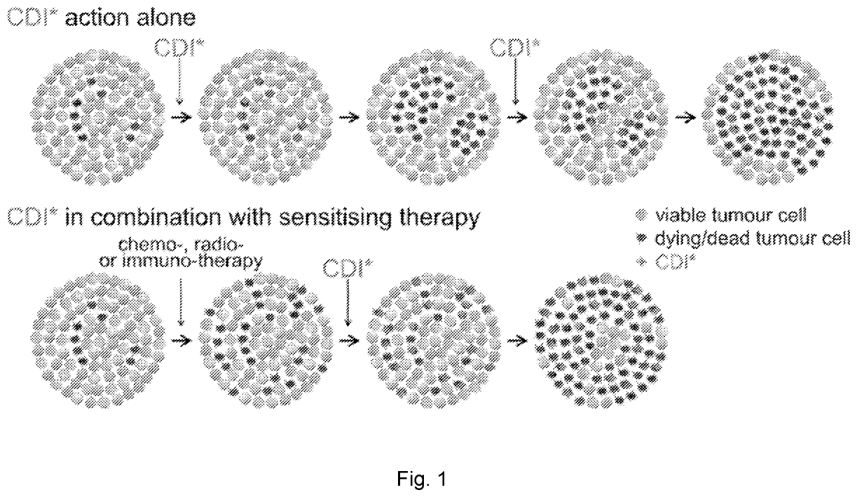

Particularly advantageously, and in contrast to other therapeutic approaches, it is possible to increase uptake of the radiolabelled compounds according to embodiments of the present disclosure by initial, concurrent or subsequent (typically initial or concurrent) administration of an initiator therapy which induces cell death, such as chemotherapy, radiotherapy, immunotherapy and/or targeted therapy, which by killing some cells, such as tumour cells, will increase uptake of radiolabelled compounds of embodiments of the present disclosure, but also have a synergistic effect with the internal radiation delivered. This mode of action is depicted in FIG. 1 (wherein ‘CDI’ refers to a radiolabelled compound comprising a therapeutic radioisotope according to the present invention). This results in a positive feedback mechanism, with a self-amplifying cascade of tumour cell kill; each cycle of treatment results in more cell death that will amplify radiolabelled compound uptake in the subsequent treatment cycle and so forth, providing exponential feedback killing of residual adjacent viable tumour cells. Compounds of the present disclosure may accordingly be delivered in multiple cycles, optionally together with multiple cycles of an initiator therapy, to provide increased cell death across the multiple cycles. An increase in cell death across the multiple cycles may include overall increases in cell death from the first to a later cycle, even if the cell death in each individual cycle is not greater than the immediate previous cycle. In some preferred embodiments, the cell death associated with an administration increases with each of the two or more cycles, i.e. the cell death associated with each administration is greater than in the immediately previous cycle. This represents a new paradigm in multimodal therapy, with the potential to transform combination therapy approaches in all malignancies. According to some embodiments, the radiolabelled compound of embodiments of the present disclosure, i.e. the therapeutic, in combination with sensitising chemotherapy, radiotherapy, immunotherapy and/or targeted therapy will generate a self-amplifying cascade of tumour cell kill—a new concept for a therapeutic.

Accordingly, the present disclosure further provides methods of treating a condition as described above, the method comprising a) optionally carrying out a treatment for said condition on a subject in need thereof; and b) administering a therapeutically effective amount of a compound described herein to the subject. The treatment of step a) is a treatment other than administering a compound or composition of the present disclosure. The treatment of step a) preferably induces some cell death, in particular in a desired location such as a tumour or cancer, or other site of a neoplastic condition. Step a) may be carried out concurrently with step b), or step b) may be carried out after step a). Steps a) and/or b) may be repeated. In some embodiments, step b) is repeated in two or more cycles, i.e. is carried out two or more times; such embodiments provide a self-amplifying treatment as discussed above. In some such embodiments, efficacy of the administration against the neoplastic condition increases across the two or more cycles, for example with each cycle, as discussed above, in particular the amount of cell death induced by each treatment comprising step a) and/or b) may increase across the two or more cycles, for example with each cycle. In some embodiments, step a) is carried out to initiate or initially increase cell death in a target area, such as a neoplastic site such as a tumour, and step b) is carried out multiple times, wherein the compound administered in step b) is taken up by dying cells resulting from step a), and further cycles of step b) further amplify the treatment effect as described above. In some embodiments, both steps a) and b) are carried out in multiple steps, whether in alternating steps or in any other order. The therapy of step a) may, in some embodiments, be selected from chemotherapy, radiotherapy, immunotherapy and targeted therapy. The present disclosure further provides compounds as described herein, comprising a therapeutic radioisotope, for use in such methods, use of said compounds in such methods, and use of said compounds in the manufacture of medicaments for treatment of the conditions described above wherein the treatment may comprise such methods.

The present disclosure further relates to a method of inducing cell death in a subject, whether in treatment of a neoplastic condition or otherwise, comprising administering a compound or a pharmaceutical composition according to the present disclosure. Such methods may be methods as described herein for treating neoplastic or other conditions, mutatis mutandis. In some embodiments, the compound or composition of the present disclosure is administered to a subject in multiple cycles, wherein the amount of cell death induced increases across the multiple cycles, for example with each cycle, due to the self-multiplying effect discussed above. Such increase in cell death may be for a given amount of compound or composition administered, relative to a previous administration.

The therapeutic compounds of the present disclosure may be used for theranostic treatment of the conditions discussed herein, by use of a therapeutic isotope which also provides emissions capable of imaging. For example, the therapeutic isotope may be a positron emitting isotope which may be imaged by use of positron emission tomography. In some embodiments, the therapeutic isotope may be ¹⁷⁷Lu, ⁶⁷Cu, ⁶⁴Cu⁹⁰Y, ¹⁸⁸Re or ¹⁸⁶Re, all of which may be imaged. A therapeutic isotope which may also be used in imaging/diagnosis allows use of the compounds of the present disclosure in theranostic methods (i.e. methods combining therapy and diagnosis/identification of target conditions such as cancers/tumours). For example, a therapeutic compound according to the present disclosure may be administered and imaging subsequently carried out to visualise where the compound has been delivered, and, in some embodiments, how much compound has been delivered, such as how much of the compound has been delivered to a target location. In some embodiments, calculation of radiation dose to tumour and normal tissue to determine probability of tumour kill and also normal tissue toxicity may be carried out with use of a theranostic compound. Since compounds of the present disclosure selectively label dying cells, visualisation of cell death by imaging of the therapeutic agent may further be used to assess changes in cell death in response to delivery of the therapeutic compounds, i.e. to monitor efficacy of the treatment. Such theranostic compounds of the present disclosure therefore allow both treatment and visualisation or monitoring of treatment with a single compound.

Therapeutic compounds of the present disclosure may also be used in combination with administration of a separate diagnostic agent, for example an imaging agent, for example an imaging agent which is targeted to neoplastic cells such as tumour cells, and which may be imaged, for example, by positron emission tomography (PET) scanning. Such a diagnostic may be used before administration of the therapeutic compounds disclosed herein, to visualise the presence of a condition, for example in the form of visualising cell death, for example in the form of tumours having high levels of cell death, and/or after treatment with compounds of the present disclosure to visualise changes in response to said treatment, for example changes in cell death. Alternatively or in addition, the diagnostic agent may be administered together with the therapeutic agent. A suitable diagnostic agent for use in such theranostic approaches is the ⁶⁸Ga labelled compound (⁶⁸Ga-NODAGA-GSAO) described in Examples 4-8 of the present application, and disclosed in PCT application PCT/AU2020/050359, the disclosure of which is incorporated herein by reference. The compounds of PCT/AU2020/050359 and methods disclosed therein may be used for imaging cell death together with treatment by administration of therapeutic compounds labelled with therapeutic radioisotopes as described herein. For example, the ⁶⁸Ga labelled compound described in the present examples may be administered before and/or after treatment with compounds labelled with therapeutic radioisotopes as described herein, and visualised by PET, to monitor efficacy of the treatment. Diagnostic compounds of PCT/AU2020/050359, in particular ⁶⁸Ga-NODAGA-GSAO, are readily synthesised, being synthesised from readily available and affordable starting materials, exhibit good biodistribution, low normal organ uptake, advantageous imaging characteristics, favourable radiation dosimetry, are non-invasive in use, and/or have a short half-life suitable for sequential repeated imaging by Positron Emission Tomography and imaging on a clinically relevant and practical timescale. The diagnostic agent may, in some embodiments, be administered intravenously.

In the context of the present disclosure, the term “diagnostic compound” may refer to a separate diagnostic compound, or to a therapeutic compound of the present disclosure which may also function as a diagnostic compound by way of imaging of the compound, i.e. a “theranostic compound”. The meaning of such terms will be readily apparent from context of use.

When administered intravenously, theranostic compounds of the present disclosure, and imaging compounds of PCT/AU2020/050359, will target dying cells and may be visualised by virtue of their radiolabelling, thus providing information on the levels of cell death in different parts of a subject, for example in response to some other therapy causing cell death. The compounds may be used to provide a measure of cell death at a single point in time, i.e. by conducting a single PET scan or other appropriate imaging technique. In some embodiments, more than one administration and/or scan may be carried out, for example, before and after a therapy is administered, to assess the changes in levels of cell death before and after therapy and to determine whether or not treatment is successful. Successful treatment of neoplastic conditions, such as a tumour, or such as cancer following administration of the therapeutic compounds of the present disclosure and/or another therapy can be determined by visualisation of increased levels of cell death at the site of the neoplastic condition by use of imageable compounds.

Diagnostic compounds, whether theranostic as described herein or a separate diagnostic compound, such as those described in PCT/AU2020/050359, may be used to tailor or alter the treatment applied, for example the intensity or duration of treatment. The measure of cell death may indicate that a therapeutic regime is or is not proving effective; where it is ineffective, an alternative dose, or an alternative treatment may be adopted. Where it is effective, treatment may be continued if required, or reduced/discontinued if required. For example the treatment dose of therapeutic compounds according to the present disclosure or some other therapy may be adjusted accordingly dependent on the level of cell death. For example, identification of patients in whom there is little or no tumour cell death following therapy would indicate the need for either an increase in the dose or duration of treatment (escalation) or a change to more intensive or multimodal therapies in order to maximise the chance of cure or disease control. Conversely, accurately assessing response early on in the course of treatment would allow a reduction in either the duration or intensity of treatment in cancer patients who are responding well in order to avoid treatment related morbidity and mortality (de-escalation) without compromising the chance of cure or disease control. An assessment of therapy success, such as by way of cell death, may cause the adoption of a new therapy, where the measure of cell death following an initial therapeutic approach suggests that the initial approach is not successful.

Use of a theranostic compound of the present disclosure comprises administering a compound of the present disclosure to a subject. Use of separate diagnostic compounds such as those disclosed in PCT/AU2020/050359 together with the therapeutic compounds of the present disclosure includes administration of an effective amount of the diagnostic compound to a subject. Such uses may further comprise conducting an imaging method on the subject following administration of the diagnostic compound (for example the theranostic compound), for example conducting PET on the subject following administration of the diagnostic compound, for example immediately after administration of the diagnostic compound. In alternative embodiments, any suitable imaging method other than PET may be used to image the diagnostic compound. In some embodiments, especially wherein the compound is a theranostic compound, nuclear medicine (gamma camera) or PET may be used to image the compound, depending on the isotope used. In some embodiments, single photon imaging (SPECT) is used to image the isotopes. The particular type of imaging suited to a given isotope and application will be readily apparent to a skilled person.

In some embodiments, PET scans are carried out after a time interval of at least 10 minutes, for example at least 20 minutes, at least 30 minutes, at least 40 minutes, at least 50 minutes, or at least 1 hour following administration of the diagnostic or theranostic compound, for example about 1 hour following administration of the diagnostic compound. In some embodiments, multiple PET scans may be carried out at various times following administration. For example, the diagnostic compound may be administered, and a PET scan may be carried out immediately following administration, as well as at about 30 minutes, about 1 hour, about 2 hours and about 3 hours following administration. Alternatively, the therapeutic compound of the present disclosure may, in some embodiments, take some time before its effects are shown; visualisation of effectiveness, such as by way of cell death, by use of a diagnostic compound or use of a theranostic compound, such as by a PET scan, may therefore take place a longer time after administration of the therapeutic or theranostic compound, for example at least or about 1 day, 3 days, 5 days, 1 week, 2 weeks or a month following administration of the therapeutic or theranostic compound. In some such embodiments wherein a separate diagnostic compound is used, a diagnostic compound may be administered prior to the scan.

In some embodiments, a method of treatment comprises administration of a therapeutic radiolabelled compound according to the present disclosure, such as for the treatment of a neoplastic condition, and administration of a separate diagnostic agent, such as disclosed in PCT/AU2020/050359, to visualise the effectiveness of the therapeutic compound, such as effectiveness in inducing cell death. The therapeutic compound may be administered to a subject together with, prior to or subsequent to administering a diagnostic compound. PET scans may be carried out following administration of the diagnostic compound to visualise the cell death-inducing activity of the therapeutic compound.

In some particular embodiments, the present disclosure provides a method of assessing a response of a subject to a treatment of a neoplastic condition, comprising: administering a therapeutic compound of the present disclosure; and visualising cell death. In some embodiments, the therapeutic compound comprises a therapeutic radioisotope capable of being imaged for visualising cell death, i.e the compound is a theranostic compound. In some embodiments, the method comprises administering a separate diagnostic compound for visualising cell death, for example as disclosed in PCT/AU2020/050359, for example ⁶⁸Ga-NODAGA-GSAO. In one particular embodiment, the cell death is visualised by conducting positron emission tomography on the subject. In one particular embodiment, cell death is visualised by way of nuclear medicine (‘gamma camera’) on the subject. In some embodiments, imaging may be carried out by single photon imaging (SPECT). In successful therapy, the assessment will show success of the therapy when a high level of cell death is visualised in the desired location. In some embodiments, a diagnostic compound is administered and/or cell death is also visualised prior to administration of the therapeutic compound, to allow comparison between the level of cell death before and after administration of the therapeutic compound. In such instances, an increase in the level of cell death between the two visualisations may indicate successful therapy. Conversely, low levels of cell death or a decrease in cell death may indicate unsuccessful or sub-optimal therapy.

In the above methods, visualisation of cell death (and optionally administration of a separate diagnostic compound) may take place, for example, about 1 day, about 2 days, about 3 days, about 1 week, about 2 weeks, about 3 weeks, about 4 weeks, about 5 weeks and/or about 6 weeks following administration of the therapeutic compound of the present disclosure. In some embodiments, visualisation of cell death takes place within 7 days of administration of the therapeutic compound. In some embodiments, visualisation of cell death takes place at least 4 weeks following administration of the therapeutic compound. In some embodiments, visualisation of cell death takes place more than once following administration of the therapeutic compound. For example, in some embodiments, visualisation of cell death takes place both within 7 days of and at least 4 weeks following administration of the therapeutic compound.

In the above methods, wherein a separate diagnostic compound is administered in order to visualise cell death, visualisation of cell death, for example by positron emission tomography, may take place, for example, at least 10 minutes, at least 20 minutes, at least 30 minutes, at least 40 minutes, at least 50 minutes, at least 1 hour or at least 90 minutes following administration of the diagnostic compound. For example, the diagnostic compound may be administered, and visualisation may be carried out, for example, immediately following administration, or about 30 minutes, about 1 hour, about 90 minutes, about 2 hours or about 3 hours following administration of the diagnostic compound.

The present disclosure relates to the above methods, compounds according to the present disclosure for use in such methods, use of compounds of the present disclosure in such methods, and use of compounds according to the present disclosure in the manufacture of a medicament for use in such methods.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The reference in this specification to any prior publication (or information derived from it), or to any matter which is known, is not, and should not be taken as, an acknowledgement or admission or any form of suggestion that prior publication (or information derived from it) or known matter forms part of the common general knowledge in the field of endeavour to which this specification relates.

The present disclosure will now be described with reference to the following specific examples, which should not be construed as in any way limiting the scope of the invention.

### EXAMPLES

### Example 1

### Synthesis of NODAGA-GSAO

a) GSAO was prepared using the process described in Park D, Don A S, Massamiri T et al (2011) Non-invasive imaging of cell death using an Hsp90 ligand. J Am Chem Soc 133:2932-3835; 4-(N-(bromoacetyl)amino)phenylarsonic acid (BRAA) was synthesized from p-arsanilic and bromoacetyl bromide, and BRAA reduced to 4-(N-(bromoacetyl)amino) phenylarsonous acid (BRAO). BRAO was coupled to glutathione (GSH) to produce GSAO. The GSAO was resolved from unreacted BRAO and GSH by C₁₈ chromatography.
b) Sodium bicarbonate and ultrapure water were purged with nitrogen for 30 minutes prior to use. The reaction setup and purification were performed under an inert atmosphere of nitrogen. GSAO obtained from step a) (20.0 mg, 36.5 μmol) was dissolved in 0.1 N sodium bicarbonate (7.4 mL) at 4° C. and stirred for 10 minutes.
c) NODAGA-NHS (2,2′-(7-(1-carboxy-4-((2,5-dioxopyrrolidin-1-yl)oxy)-4-oxobutyl)-1,4,7-triazonane-1,4-diyl)diacetic acid mono-N-hydroxysuccinimide ester) obtained from CheMatech (Dijon, France) (34.5 mg, 47.0 μmol) was dissolved in anhydrous dimethylformamide (DMF) (1 mL) and added to the reaction mixture obtained in step b) dropwise over 1 hour.
d) The reaction mixture was stirred for 4 hours, acidified by the addition of 1 M hydrochloric acid (1 mL), shock-frozen in liquid nitrogen, and freeze-dried.

### NODAGA-GSAO Purification

e) The residue resulting from step d) was redissolved in deaerated water (4 mL), filtered (0.45 μm), and purified by reverse phase high-performance liquid chromatography (RP-HPLC). A gradient of 2-20% mobile phase B (0.2% trifluoroacetic acid (TFA) in acetonitrile) in mobile phase A (0.2% TFA in ultrapure water) was applied from 0 to 25 minutes. NODAGA-GSAO was eluted at 20.6 minutes. The sample was collected by hand and each fraction was instantly purged with nitrogen.

HPLC was carried out on a Shimadzu LC-20 series LC system with two LC-20AP pumps, a SIL-1OAP autosampler, an SPD-20A UV/VIS detector, and a Shimadzu ShimPack GIS-C18 column (150×10.0 mm i.d., 5 μm, 4 mL/mini) (System A). Shimadzu LabSolutions Software (Ver. 5.73) was used for data acquisition and processing.
f) The pooled fractions were frozen at −20° C. and freeze-dried to give 7.3 mg of white powder (21.6% yield).
g) NODAGA-GSAO was dispensed in aliquots of 54 μg per 100 μL water and stored at −80° C.
h) The purity of the compound (>95%) was verified by injecting a solution of NODAGA-GSAO (5 μL; approx. 17 mM, in water) onto liquid chromatography-mass spectrometry (LC-MS) at 2-2-50% mobile phase B (0.1% formic acid (FA) in acetonitrile) in mobile phase A (0.1% FA in mass spectrometry-grade water) over 0-5-45 minutes. NODAGA-GSAO eluted at 19.4 minutes.

LC-MS was conducted using an Agilent system (Santa Clara, CA, USA) consisting of a 1260 series quarternary pump with an inbuilt degasser, 1200 series autosampler, thermostated column compartment, diode array detector, fraction collector, a 6120 series single-quadrupole mass spectrometer, and an Agilent Zorbax Eclipse XDB-C18 column (150×4.6 mm i.d., 5 μm) at 30° C. (System B). The drying gas flow, temperature, and nebulizer were set to 12 L/min, 350° C., and 35 psi respectively. Agilent OpenLAB Chromatography Data System (CDS) ChemStationEdition (C.01.05) was used for data acquisition and processing. Electrospray ionization (ESI) was used to analyse aliquots (5 μL) in positive ion mode with a 3500 V capillary voltage. Nuclear magnetic resonance (NMR) spectroscopy (¹H and ¹³C) spectra were recorded in 5 mm Pyrex tubes (Wilmad, USA) using a Varian 400-MR NMR spectrometer (Lexington, MA, USA) at a frequency of 399.73 MHz (H) or 100.51 MHz (¹³C) at 24° C. operated with VnmrJ 3.1 software (Agilent Technologies, Santa Clara, CA, USA). The spectral data are reported in ppm (δ) and referenced to residual solvent (deuterated dimethyl sulfoxide [DMSO-d₆] 2.50/39.52 ppm).
i) Absorbance was measured at 210 and 254 nm and the respective area under the curve (AUC) was used to determine compound purity as a percent of total AUC compared to background.

### Example 2

Labelling of NODAGA-GSAO with ¹⁷⁵Lutetium (¹⁷⁵Lu), ⁶³Copper (⁶³Cu) and ⁸⁹Yttrium (⁸⁹Y)

Before labelling with radioisotopes, binding conditions using stable isotopes were evaluated. In particular, ¹⁷⁵Lu, ⁶³Cu and ⁸⁹Y were used in place of ¹⁷⁷Lu, ⁶⁷Cu and ⁹⁰Y respectively. These experiments determined the optimal binding conditions and function as proof of concept for the radioisotopes.

Labelling of NODAGA-GSAO (62 μM) obtained in Example 1 was carried out in 0.4 M sodium acetate (Sigma Aldrich) buffer at various pH levels and temperatures, as indicated below. Stable isotope ¹⁷⁵Lu (as lutetium (III) chloride), ⁶³Cu (as copper(II) sulphate pentahydrate) or ⁸⁹Y (yttrium(III) chloride) (Sigma Aldrich) were added at a 1.2-fold molar ratio relative to NODAGA-GSAO and the mixture incubated for 30 minutes.

All experiments were performed on an Agilent (Santa Clara, CA, USA) 1260 Infinity Quaternary LC and analysed using the Agilent OpenLab CDS ChemStation Edition software. The analytical column was an Alltima HP C18 150×4.6 mm 5 μm particle size (Hichrom, Berkshire, UK). The column was equilibrated in a mixture of 0.1% (v/v) trifluoroacetic acid (Sigma Aldrich) in MiliQ water (mobile phase A) and acetonitrile (mobile phase B; 98/2, v/v) (Unichrom, Thermo Fisher Scientific). Samples (100 μL) were loaded on the column using the autosampler at room temperature and products were eluted using a gradient of 2-20-70-2-2% mobile phase B in mobile phase A over 0-18-28-28-33 min at a flowrate of 0.6 mL/min. Absorbance was measured at 210 nm and 280 nm.

The extent of labelling was determined as the percentage of the area under the curve (AUC) of the labelled CDI peak (time to peak, 13.9-14.1) over the total AUC compared to background.

The pH-dependent labelling of NODAGA-GSAO with stable isotopes following incubation at room temperature (63Cu), 80° C. (89Y), or 85° C. (175Lu) for 30 min is shown in Table 1 below. Data are from one or two (average±S.D. of the two measurements) experiments.

TABLE 1

| | Labelling (%) | | | | |
|---|---|---|---|---|---|
| Isotope | pH 4.0 | pH 4.5 | pH 5.0 | pH 5.5 | pH 6.0 |
| ⁶³Cu | >99.9 ± 0.0 | >99.9 ± 0.0 | >99.9 ± 0.0 | >99.9 ± 0.0 | >99.9 ± 0.0 |
| ⁸⁹Y | 18.59 | 44.46 | 62.69 | 58.33 | 57.72 |
| ¹⁷⁵Lu | 77.34 | 90.05 | 92.08 | 91.52 | 92.21 |

Time- and temperature-dependent labelling of NODAGA-GSAO with ¹⁷⁵Lu or ⁸⁹Y at pH 5 is shown in Table 2 below (N.D.=not determined). Data are from one or two (average±S.D. of the two measurements) experiments.

TABLE 2

| | Reaction | | | |
|---|---|---|---|---|
| | temperature | Labelling (%) | | |
| Isotope | and pH | 10 min | 20 min | 30 min |
| ¹⁷⁵Lu | RT, pH 5.0 | N.D. | N.D. | 7.76 |
| | 60° C., pH 5.0 | 53.02 | 70.57 | 76.00 |
| | 70° C., pH 5.0 | 79.13 | 90.16 | 91.27 |
| | 80° C., pH 5.0 | 91.49 | 92.12 ± 0.26 | 94.08 ± 0.18 |
| | 85° C., pH 5.0 | N.D. | N.D. | 92.08 |
| | 90° C., pH 5.0 | 93.88 | 91.37 | 90.24 |
| | 100° C., pH 5.0 | 92.94 | 94.13 | 90.81 |
| | 110° C., pH 5.0 | 92.50 | 90.06 | 90.29 |
| | 120° C., pH 5.0 | 90.85 | 90.07 | 90.46 |
| ⁸⁹Y | RT, pH 4.5 | N.D. | N.D. | 11.63 |
| | 80° C., pH 4.5 | N.D. | N.D. | 44.46 |
| | 80° C., pH 5.0 | N.D. | N.D. | 62.69 |
| | 120° C., pH 5.0 | N.D. | N.D. | 57.52 |

### ¹⁷⁵Lu

Binding of ¹⁷⁵Lu with NODAGA-GSAO was found to be suboptimal at pH 4.0 or 4.5, but efficient at pH≥5.0. Binding at pH 5.0 was found to be inefficient (7.76%) with 30 minutes incubation at room temperature, but increasing the temperature to 60-80° C. enhanced labelling in a temperature- and time-dependent manner, and maximum labelling was found following a 30 minute incubation at 80° C.

To ensure that the observed elution peaks constituted ¹⁷⁵Lu-NODAGA-GSAO, the labelled products were pre-incubated with DMP for 15 min at room temperature and assessed by HPLC. The dithiol of DMP engages the As(III) hydroxyl groups of NODAGA-GSAO to form a five-membered ring structure that right-shifts the elution peak, as shown in Scheme 2 below.

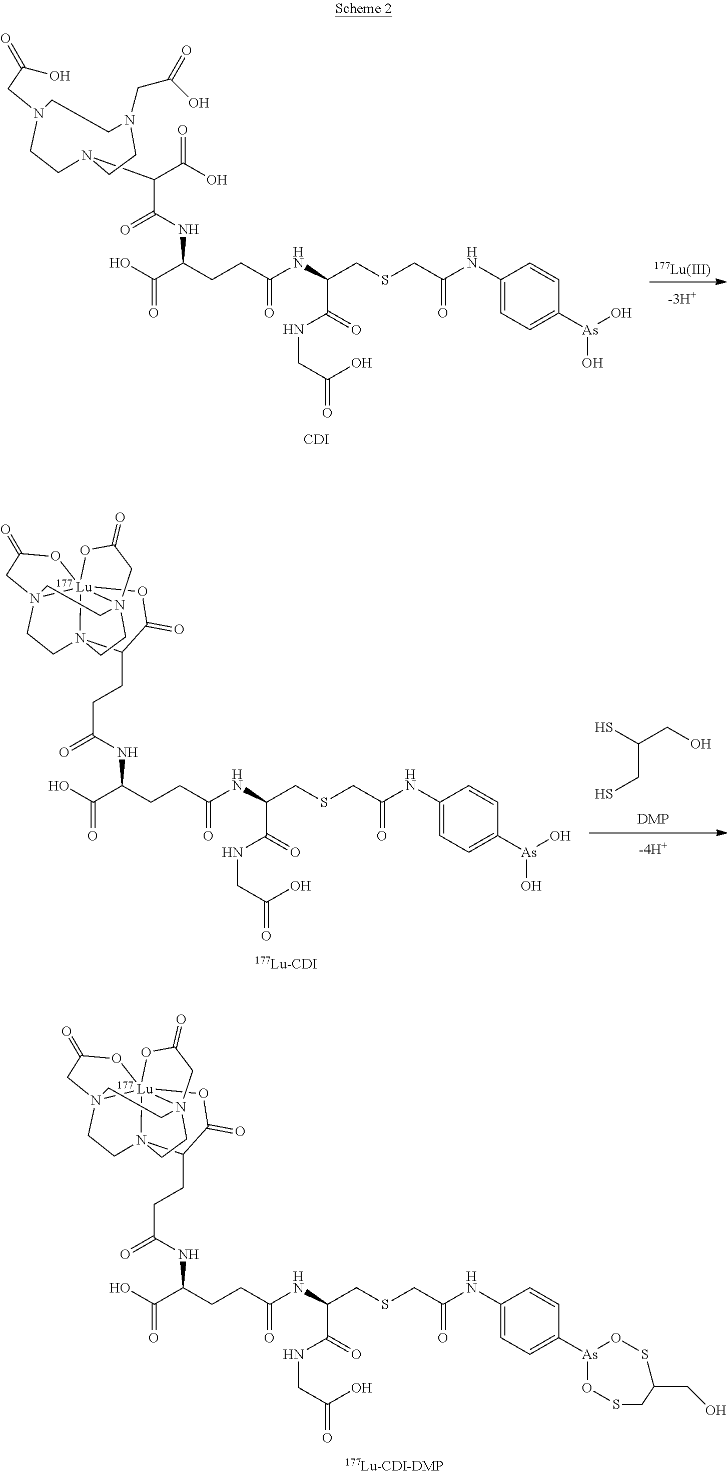

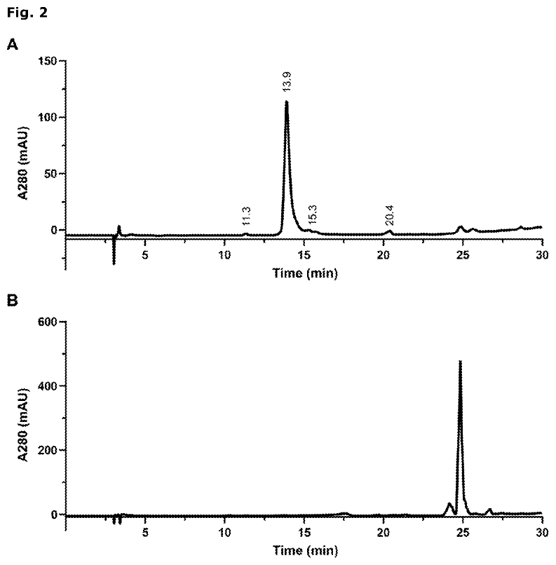

The HPLC chromatogram of ¹⁷⁵Lu-NODAGA-GSAO labelled for 30 minutes at pH 5.0 at 80° C. is shown in FIG. 2, (A) without DMP or (B) with pre-incubation with DMP as described above. Time to peak of relevant elution peaks are indicated. Data are representative of 2 independent experiments. Incubation of ¹⁷⁵Lu-NODAGA-GSAO with DMP shifted the time for elution of the compound from 13.9 to 25.0 min, confirming that the compound contains active As(III) targeting moiety.

### ⁶³Cu

Labelling with ⁶³Cu was found to be near 100% for all pH conditions tested with incubation at room temperature.

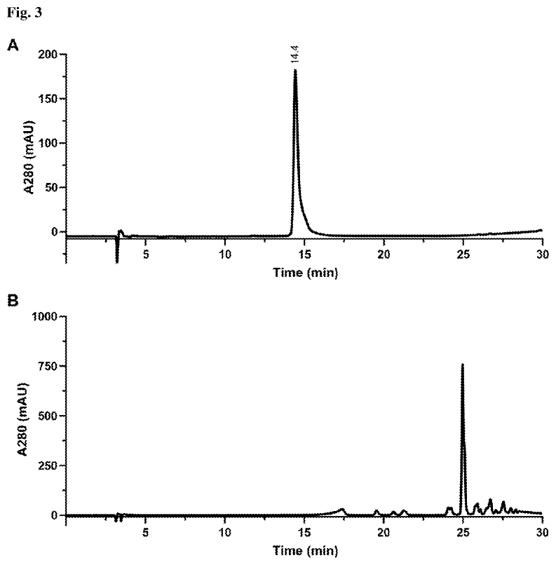

Incubation of the ⁶³Cu-NODAGA-GSAO product with DMP confirmed that it contained active As(III). Chromatograms of ⁶³Cu-NODAGA-GSAO by HPLC labelled for 30 minutes at pH 5.0 at room temperature is shown in FIG. 3, (A) without DMP or (B) with pre-incubation with DMP as described above.

### ⁸⁹Y

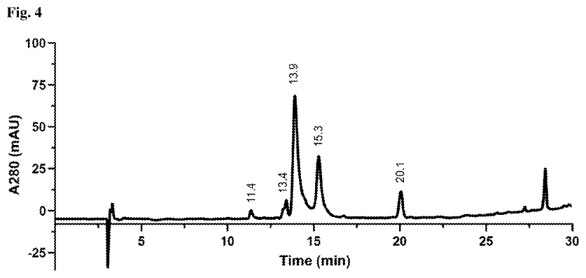

With ⁸⁹Y, labelling was found to be ineffective at pH 4.0 or 4.5 and reached maximal values at pH≥5.0, up to about 60% NODAGA-GSAO labelling, at 80° C. A modest increase in labelling at 120° C. occurred, but by-product generation was observed at 120° C. An HPLC chromatogram of ⁸⁹Y-NODAGA-GSAO labelled for 30 minutes at pH 5.0 at 120° C. is shown in FIG. 4.

### Example 3

### Stability of Labelled NODAGA-GSAO Products

The in vitro stability of the complexed product is an important determinant for the potential clinical use of a therapeutic compound. Post-labelling stability was assessed by incubation of the ¹⁷⁵Lu- and ⁶³Cu-labelled NODAGA-GSAO products at room temperature.

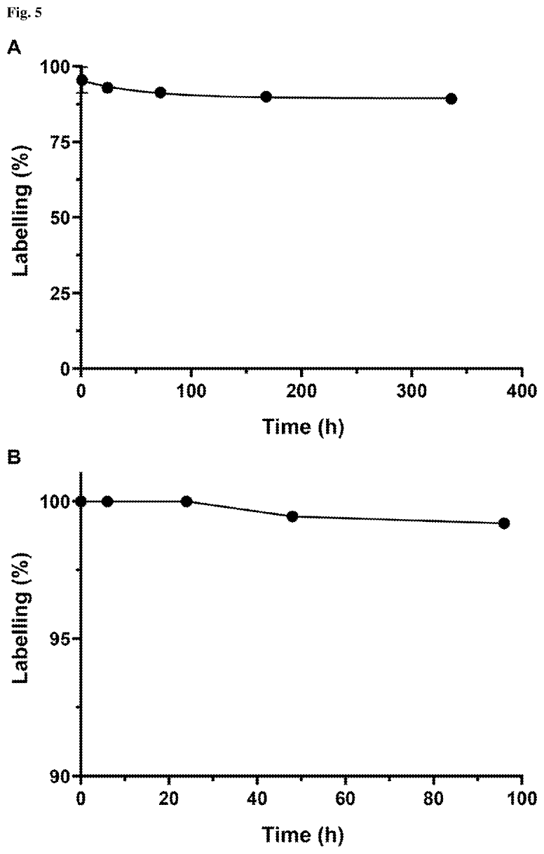

The percentage of labelling at different time-points following formation of the isotope-NODAGA-GSAO complex was measured. Results are shown in FIG. 5 for A) the ¹⁷⁵Lu-labelled product obtained from incubation for 30 minutes at pH 5.0 at 80° C. and B) the ⁶³Cu-labelled product obtained from incubation for 30 minutes at pH 5.0 at room temperature. At certain time points, aliquots were taken and the labelling (black circles) of NODAGA-GSAO were assessed by HPLC. Data are from one or two (average±S.D. of the two measurements) experiments.

For ¹⁷⁵Lu-NODAGA-GSAO, the labelling slightly decreased over time but remained ˜90% after 14 days of storage. In addition, the loss of labelling coincided with increased levels of by-products (4-7.5% of total AUC).

NODAGA-GSAO chelated with ⁶³Cu was highly stable for up to four days and only a minor amount of a single by-product was formed (<1% of total AUC). The by-products observed with ¹⁷⁵Lu-NODAGA-GSAO but not ⁶³Cu-NODAGA-GSAO are likely due to the heating during reaction with ¹⁷⁵Lu. Considering the half-life of the radioisotopes and the time of treatment following product synthesis, ¹⁷⁵Lu- and ⁶³Cu-labelled NODAGA-GSAO display sufficient stability to pursue therapeutic evaluation.

The above results demonstrate that NODAGA-GSAO can be efficiently labelled with isotopes of Lu and Cu; therapeutic radioisotopes with an established clinical role in radiation oncology. Furthermore, high in vitro stability of ¹⁷⁵Lu- and ⁶³Cu-labelled NODAGA-GSAO has been demonstrated. By employing the unique properties of NODAGA-GSAO, these conjugates provide a promising therapeutic approach for targeting dying and dead tumour cells and provides a novel means of delivering therapeutic radiation to adjacent viable tumour cells.

### Example 4

Labelling of NODAGA-GSAO with ¹⁷⁷Lutetium (¹⁷⁷Lu)

Following labelling of NODAGA-GSAO with stable isotopes as described in Example 2, NODAGA-GSAO was successfully labelled with radioisotope ¹⁷⁷Lu at a specific activity of 500 MBq/54 μg NODAGA-GSAO (˜2 GBq/216 μg NODAGA-GSAO).

### Synthesis

First, [¹⁷⁷Lu]LuCl₃ was diluted to form a stock solution. 1.0 mL of 0.04 M HCL was added to a vial of 0.5 mL ¹⁷⁷LuCl (no carrier added) (ANSTO). All [¹⁷⁷Lu]LuCl₃ was then transferred to into a 10 mL evacuated vial. A further 1.5 mL of 0.04 M HCl was used to rinse the residual [¹⁷⁷Lu]LuCl₃ into the evacuated vial to give a solution of [¹⁷⁷Lu]LuCl₃ with 5.0 GBq in 5.0 mL (radioactive concentration=1 GBq/mL). A vent needle with syringe attached was inserted to equilibrate the pressure.

A vial of NODAGA-GSAO prepared as Example 1 (54 μg in 100 μL, 0.06 μmol) was thawed and the contents pipetted into an Eppendorf tube. 100 μL of 0.25 M ascorbic acid was then added, followed by 250 μL of sodium acetate binding buffer (CH₃COONa·3H₂O, 1.5 M, pH 4.5, MW 136.08). 0.25 M ascorbic acid was obtained by dissolving 44 mg ascorbic acid (Merck 100468) in 1 mL Ultrapure water. The overall concentration of ascorbic acid in the reaction mixture was 0.0056 M.

The sodium acetate buffer was obtained by dissolving 10.21 g CH₃COONa·3H₂O (Merck 106267) in 40 mL Ultrapure water, adjusting to pH 5.0 with glacial acetic acid, and adding Ultrapure water to give a total volume of 50 mL.

Ultrapure water was then drawn into a syringe so that the total volume of water, NODAGA-GSAO, ascorbic acid, and binding buffer (and ethanol or glutathione when used in Example 5 below) was 4 mL. The filled syringe was then used to draw the contents of the Eppendorf tube and transfer into an evacuated vial. 500 μL of ¹⁷⁷LuC₃ solution was then added. A vent needle was inserted to equilibrate pressure with a syringe, and the vial wrapped with Parafilm to prevent aerosol contamination. The vial was then incubated for 30 minutes at 85° C.

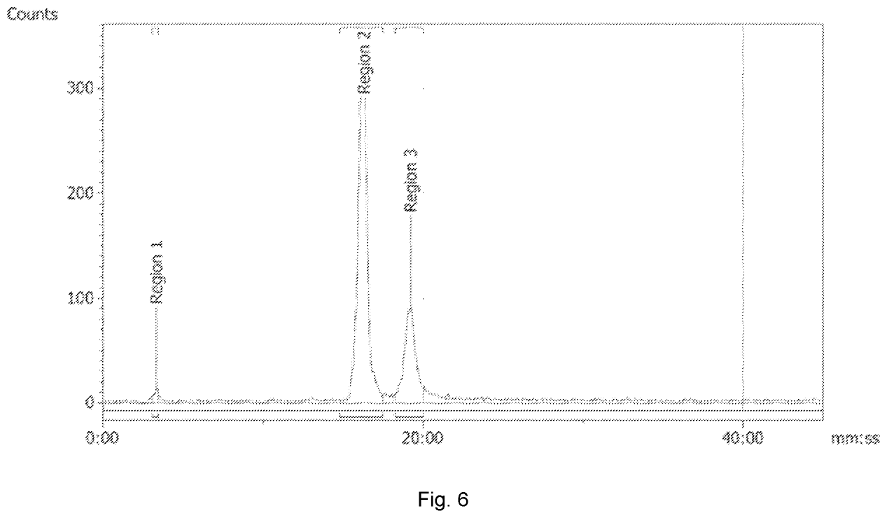

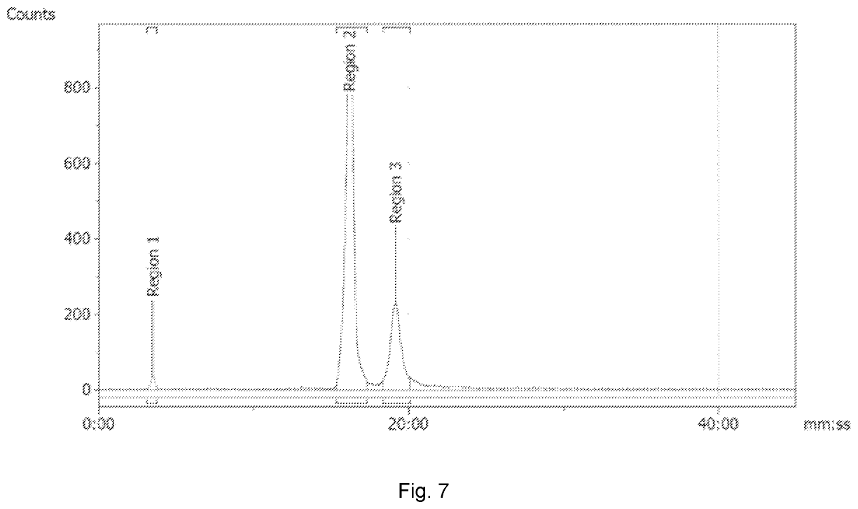

Approximately 0.4 mL of the reaction vial contents was then withdrawn for analysis. 200 μL was placed into an autosampler vial with insert and HPLC analysis performed. 200 μL was also placed into an autosampler vial with insert containing 10 μL DMP:DMSO and HPLC analysis performed. DMP:DMSO was obtained by dissolving 5 μL 2,3-Dimercapto-1-propanol (DMP) (Sigma D1129) in 495 μL DMSO). Results are shown in FIGS. 6 and 7. pH was also measured using paper.

### Post-Synthesis Purification

The contents of the reaction vial was withdrawn into a syringe and loaded onto an Oasis PRiME HLB cartridge (335 mg sorbent, primed with 1 mL ethanol and 10 mL water for injection) at approximately ˜1 mL·min⁻¹, purged with air and waste collected in a waste vial. The reaction vial was further rinsed with 10 mL normal saline and load onto the Oasis PRiME HLB cartridge at approximately ˜1 mL·min⁻¹, purged with air and waste collected in the waste vial.

The product was eluted off the Oasis PRiME HLB cartridge with 0.5 mL ethanol and purged with air, collecting product into product vial. The Oasis PRiME HLB cartridge was further rinsed with 9.5 mL normal saline at approximately ˜1 mL·min⁻¹ and purged with air, collecting into a product vial.

Activity was measured in the waste vial, Oasis PRiME HLB cartridge and product vial.

Approximately 0.4 mL of the product vial was withdrawn for analysis. Approximately 200 μL was placed into an autosampler vial with insert and HPLC analysis performed. Approximately 200 μL was also placed into an autosampler vial with insert containing 10 μL DMP:DMSO and HPLC analysis performed (2,3-Dimercapto-1-propanol (DMP) (Sigma D1129) solution for HPLC was obtained by dissolving 5 μL DMP in 495 μL DMSO), as above. pH was also measured using paper.

HPLC parameters are provided below:

Solutions
A: trifluoroacetic acid (TFA)/H₂O
B: acetonitrile (ACN)
C: H₂O
D: MeOH

Gradients

TABLE 3
Analysis gradient

| Time | Flow rate | % A (H₂0/ | % B |
|---|---|---|---|
| (min) | (mL) | 0.1% TFA) | ACN |
| 0 | 0.6 | 98 | 2 |
| 18 | 0.6 | 80 | 20 |
| 28 | 0.6 | 30 | 70 |
| 38 | 0.6 | 98 | 2 |
| 42 | 0.6 | 98 | 2 |

TABLE 4
Clean Gradient

| | Flow | | |
|---|---|---|---|
| Time | rate | % C (H₂0/ | % D |
| (min) | (mL) | 0.1% TFA) | MeOH |
| 0 | 1.0 | 95 | 5 |
| 5 | 1.0 | 95 | 5 |
| 25 | 1.0 | 5 | 95 |
| 30 | 1.0 | 5 | 95 |
| 40 | 1.0 | 50 | 50 |
| 45 | 1.0 | 50 | 50 |

### Results

NODAGA-GSAO was successfully labelled with ¹⁷⁷Lu at 500 MBq/˜51 μg NODAGA-GSAO. Radiochromatograms of the reaction product before post-synthesis purification are shown in FIG. 6 and FIG. 7, of the reaction product at the end of synthesis (FIG. 6) and at 1.5 hours after the end of synthesis (FIG. 7). Region 2 is oxidised NODAGA-GSAO. Region 3 is ¹⁷⁷Lu-NODAGA-GSAO.

The chromatograms show that very low amounts of free Lu-177 are present in the reaction product, even before any post-synthesis purification is carried out. Whilst NODAGA-GSAO was successfully labelled with ¹⁷⁷Lu as described above, radiolysis occurred during synthesis, producing oxidised NODAGA-GSAO, as shown in FIGS. 6 and 7. Following synthesis, no further radiolysis occurs, suggesting that both heat and free radical generation are required for radiolysis. Accordingly, methods for reducing radiolysis were investigated as described below.

### Example 5

Labelling of NODAGA-GSAO with ¹⁷⁷Lutetium (¹⁷⁷Lu) with Reduction of Radiolysis

As demonstrated in Example 4 above, presence of ascorbic acid during labelling of NODAGA-GSAO with ¹⁷⁷Lu incompletely prevented radiolysis. Several methods for further prevention of radiolysis were investigated:

### a) Ethanol

The method as described in Example 4 was used to prepare ¹⁷⁷Lu-NODAGA-GSAO, except that during synthesis, 100 μL of ethanol was added to NODAGA-GSAO instead of 100 μL M ascorbic acid.

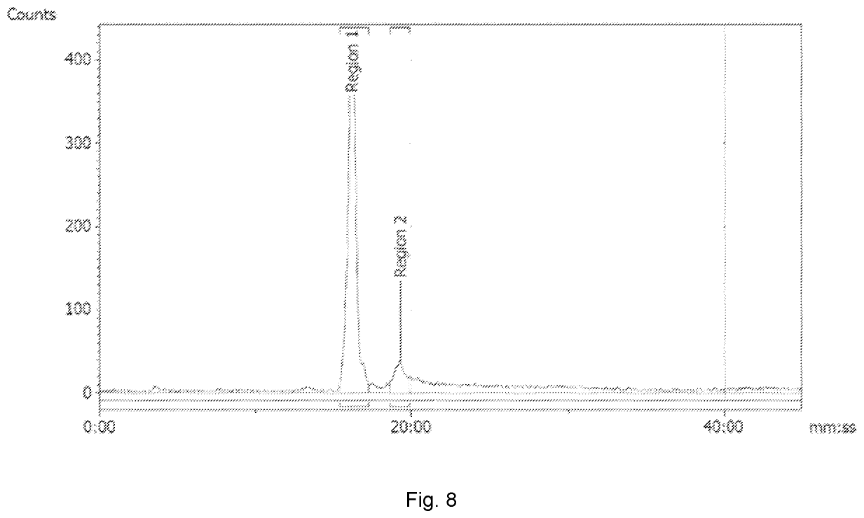

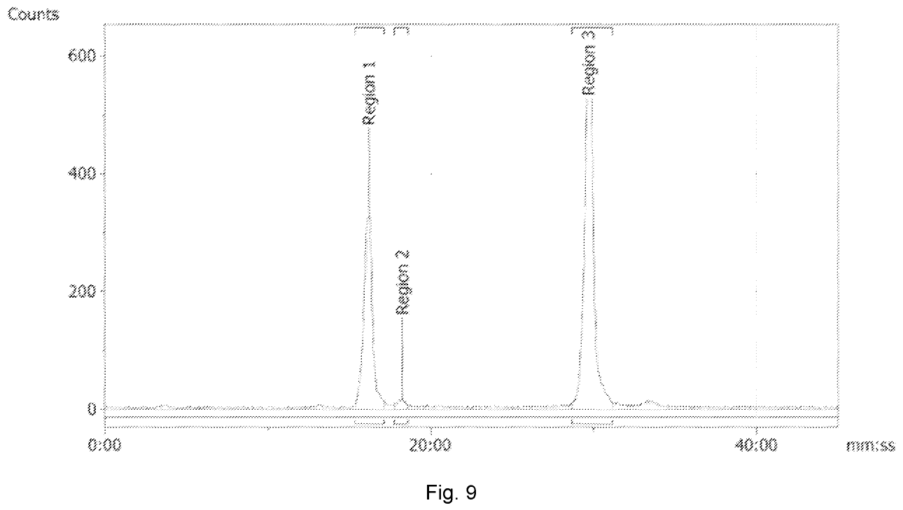

Radiochromatograms of the reaction product before post-synthesis purification are shown in FIGS. 8 and 9. A radiochromatogram of the reaction at the end of synthesis is shown in FIG. 8. A radiochromatogram of the product mixed with 1% DMP in DMSO is shown in FIG. 9. Region 1 is oxidised NODAGA-GSAO. Region 2 is ¹⁷⁷Lu-NODAGA-GSAO. Region 3 is a cyclic dithioarsinite complex of DMP with the As(III) atom of NODAGA-GSAO.

As can be seen from the chromatograms, radiolabelling was achieved in the presence of ethanol. However, ethanol provided minimal protection from radiolysis. A proportion of the reaction was still able to form a cyclic dithioarsinite complex with DMP.

### b) High Concentration Ascorbic Acid

The method as described in Example 4 was used to prepare ¹⁷⁷Lu-NODAGA-GSAO, except that during synthesis, 500 μL of 0.25 M ascorbic acid instead of 100 μL ascorbic acid was added to NODAGA-GSAO. The overall concentration of ascorbic acid in the reaction mixture was 0.023 M.

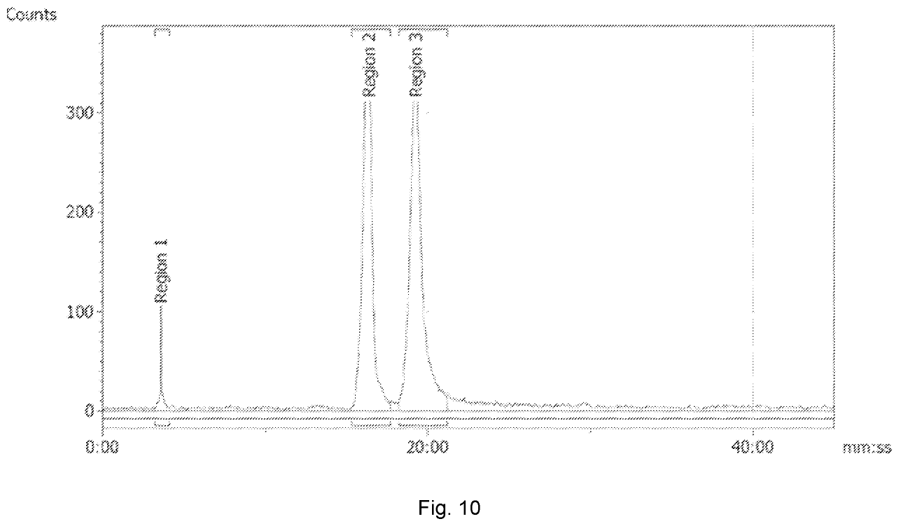

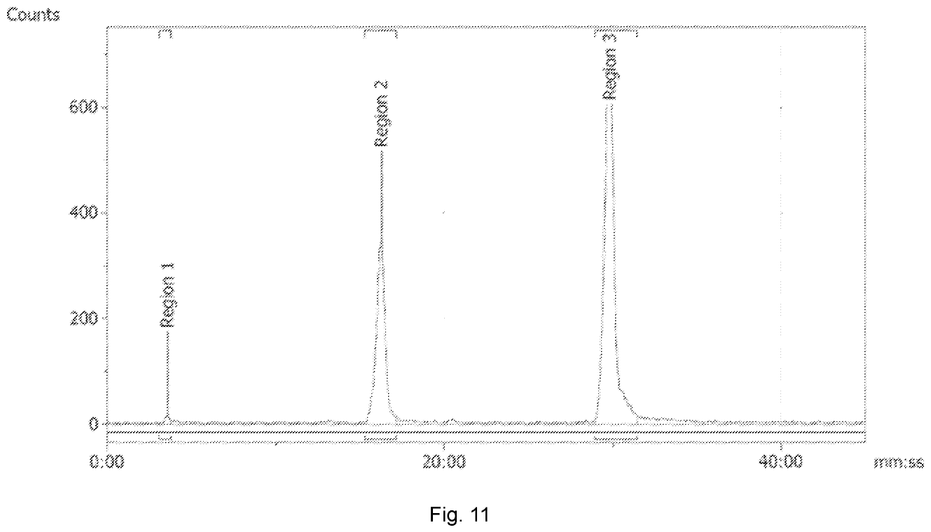

Radiochromatograms of the reaction product before post-synthesis purification are shown in FIGS. 10 and 11. A radiochromatogram of the reaction at the end of synthesis is shown in FIG. 10. Region 2 is oxidised NODAGA-GSAO. Region 3 is ¹⁷⁷Lu-NODAGA-GSAO. A radiochromatogram of the product mixed with 1% DMP in DMSO is shown in FIG. 11. Region 2 is oxidised NODAGA-GSAO. Region 3 is a cyclic dithioarsinite complex of DMP with the As(III) atom of NODAGA-GSAO.

As seen in the radiochromatograms, increasing amounts of ascorbic acid provided some additional protection against radiolysis.

### c) High Concentration Ascorbic Acid and Glutathione

The method as described in Example 4 was used to prepare ¹⁷⁷Lu-NODAGA-GSAO, except that during synthesis, 500 μL of ascorbic acid instead of 0.2 M 100 μL ascorbic acid was added to NODAGA-GSAO, as well as 500 μL 0.25 M glutathione (obtained by dissolving 77 mg L-Glutathione Reduced (Sigma G4251-25G) in 1 mL Ultrapure water). The overall concentration of each of the glutathione and the ascorbic acid in the reaction mixture was 0.023 M.

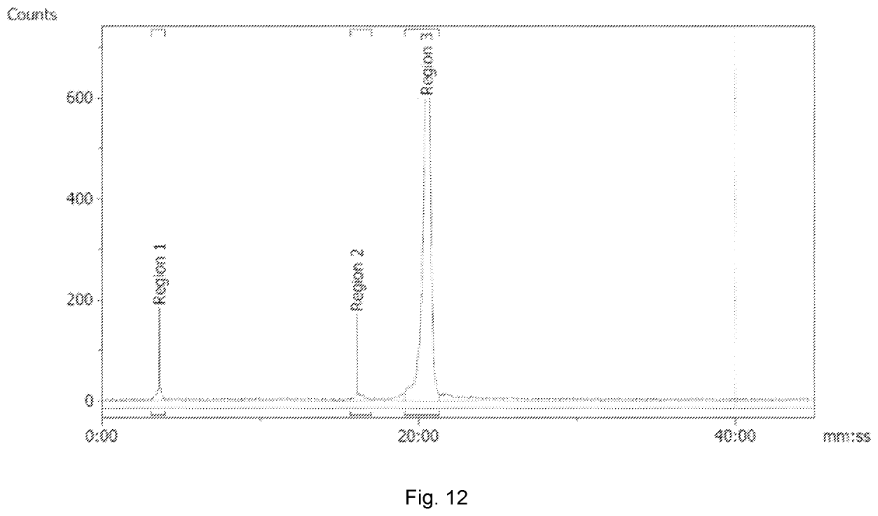

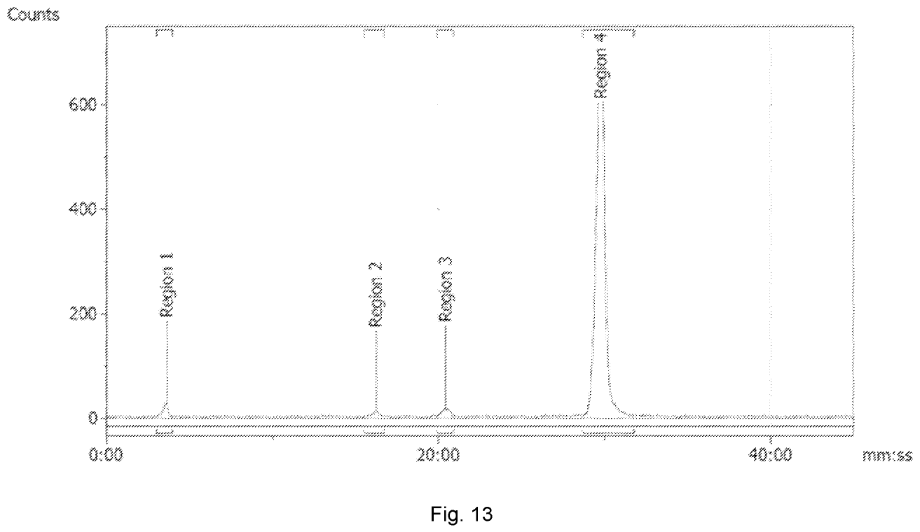

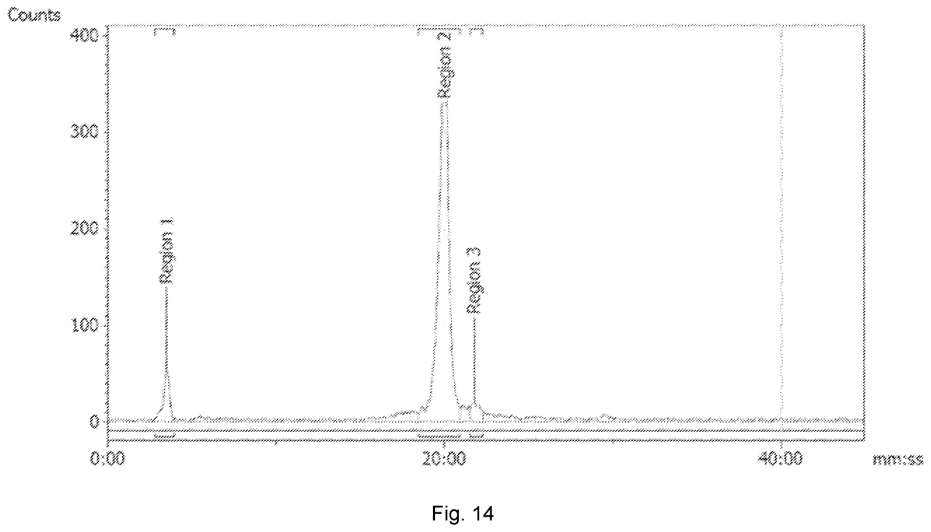

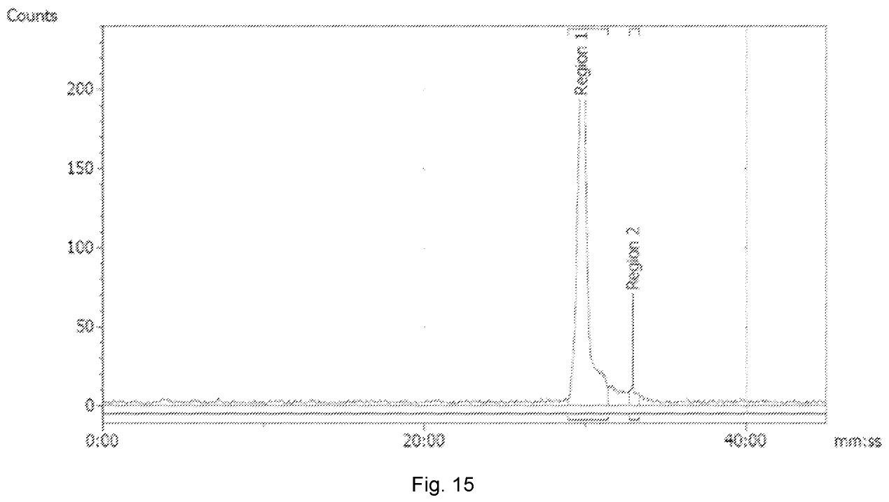

Radiochromatograms of the reaction product before post-synthesis purification are shown in FIGS. 12-15. A radiochromatogram of the reaction at the end of synthesis is shown in FIG. 12. Region 2 is oxidised NODAGA-GSAO. Region 3 is ¹⁷⁷Lu-NODAGA-GSAO. A radiochromatogram of the product at the end of synthesis mixed with 1% DMP in DMSO is shown in FIG. 13. Region 2 is oxidised NODAGA-GSAO. Region 3 is a cyclic dithioarsinite complex of DMP with the As(III) atom of NODAGA-GSAO. A radiochromatogram of the product at 72 hours post synthesis is shown in FIG. 14. Region 2 is ¹⁷⁷Lu-NODAGA-GSAO. A radiochromatogram of the product at 72 hours post synthesis mixed with 1% DMP in DMSO is shown in FIG. 15. Region 1 is a cyclic dithioarsinite complex of DMP with the As(III) atom of NODAGA-GSAO.

As seen in the radiochromatograms, a combination of high concentration of ascorbic acid and glutathione almost completely prevented radiolysis, both during and up to 72 hours post-synthesis. Importantly, ascorbic acid and glutathione are biocompatible compounds.

### d) Reduced Concentration of Glutathione

The method as described in Example 5c) above was used to prepare ¹⁷⁷Lu-NODAGA-GSAO, except that during synthesis, 100 μL instead of 500 μL 0.25 M glutathione was used. The overall concentration of glutathione in the reaction mixture was 0.0056 M.

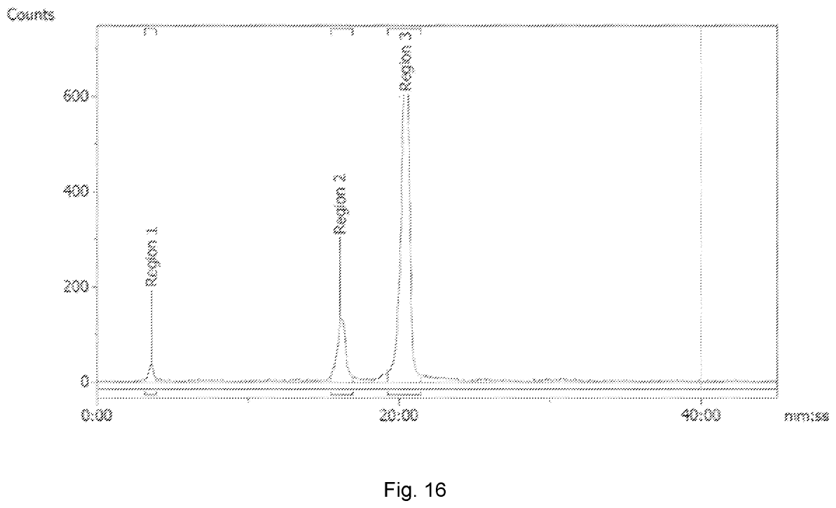

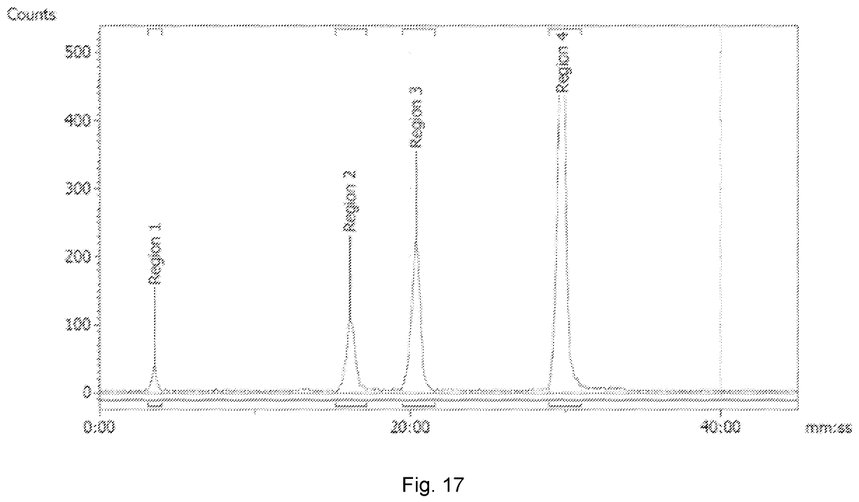

Radiochromatograms of the reaction product before post-synthesis purification are shown in FIGS. 16 and 17. A radiochromatogram of the reaction at the end of synthesis is shown in FIG. 16. Region 2 is oxidised NODAGA-GSAO. Region 3 is ¹⁷⁷Lu-NODAGA-GSAO. A radiochromatogram of the product mixed with 1% DMP in DMSO is shown in FIG. 17. Region 2 is oxidised NODAGA-GSAO. Region 3 is ¹⁷⁷Lu-NODAGA-GSAO. Region 4 is a cyclic dithioarsinite complex of DMP with the As(III) atom of NODAGA-GSAO.

Reducing the concentration of glutathione resulted in an increase in radiolysis of NODAGA-GSAO. Additionally, there was a component of NODAGA-GSAO which appeared not to form a cyclic dithioarsinite complex of DMP with the As(III) atom.

### Example 6

Radiolabelling of NODAGA-GSAO with ⁶⁸Ga

⁶⁸Ga was used to radiolabel NODAGA-GSAO in place of a therapeutic isotope, as described in PCT application PCT/AU2020/050359 and depicted in Scheme 3 below. Such compounds are useful in imaging of cell death, for example for monitoring the progress of a condition associated with cell death, for example a neoplastic condition such as a tumour or cancer, or for monitoring effectiveness of a treatment. Such imaging may be carried out for example by way of positron emission tomography.

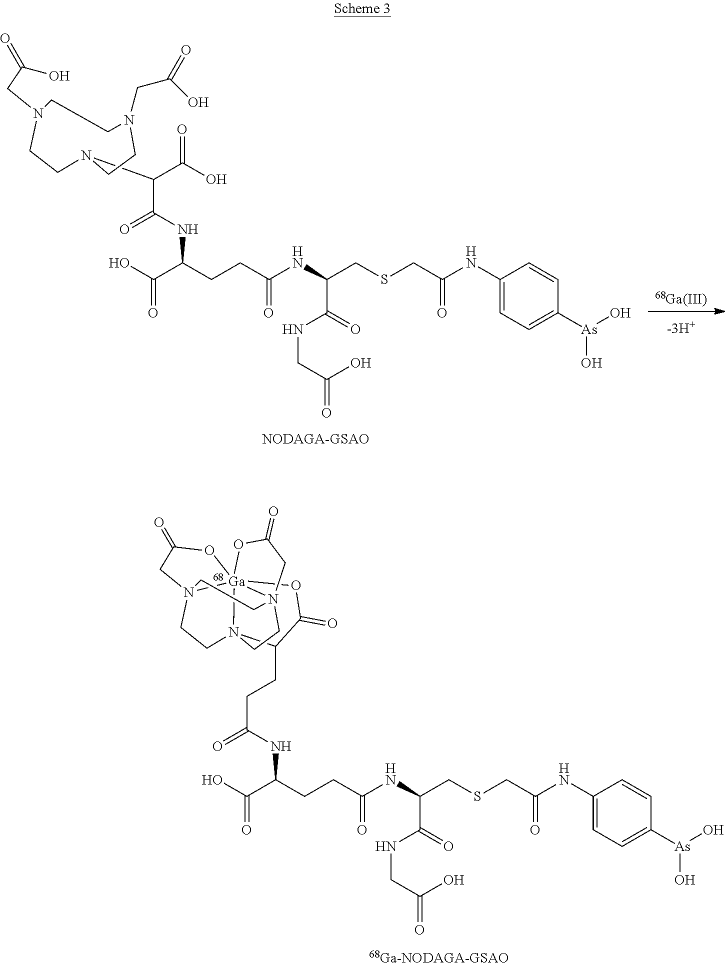

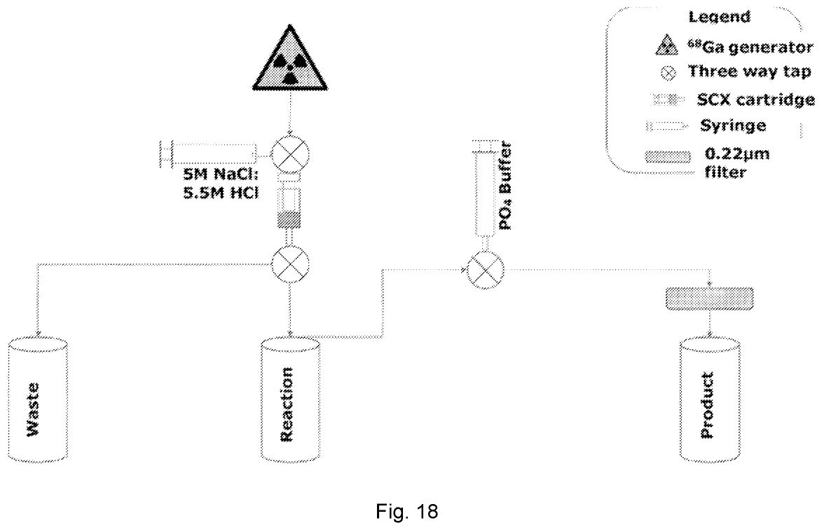

The methods and procedures of the following Examples described in relation to ⁶⁸Ga may be applied to compounds comprising a therapeutic radioisotope as described herein mutatis mutandis. However, labelling with ¹⁷⁷Lu or ⁶⁷Cu may be carried out without steps a) to c), g) and h) and below (i.e. without use of a SCX column; the radioisotope is added to the NODAGA-GSAO without initial cation exchange).
a) The barrel of a BondElute SCX column was cut so that when inserted the barbed female luer thread rested just above the column media to create a cartridge (hereafter referred to as the SCX cartridge). The barbed female luer thread should be fitted firmly and securely into the cut barrel of the BondElute SCX column to create a sealed cartridge that is air- and liquid-tight.
b) The SCX cartridge was primed with 1 mL 5.5 M HCl and then flushed with 10 mL of water.
c) The SCX cartridge was purged with air.
d) Ascorbic acid solution (0.25 M) was obtained by dissolving 44 mg of ascorbic acid in 1 mL water (Water Ultrapur, Merck).
e) A sodium acetate buffer (1.5 M CH₃COONa·3H₂O, pH4.5) was obtained by dissolving 10.21 g CH₃COONa·3H₂O in water (Water Ultrapur, Merck). The pH was adjusted to pH 4.5 with glacial acetic acid and water was added to a total volume of 50 mL.
f) One vial of 54 μg NODAGA-GSAO obtained in Example 1 was thawed and mixed with 100 μL of ascorbic acid solution (used as a free radical scavenger since GSAO is sensitive to radiolysis and oxidation), 250 μL of sodium acetate buffer, and 3.5 mL water and the mixture transferred to a 10 mL evacuated glass reaction vial.
g) The ⁶⁸Ga was eluted according to the supplier's instructions onto the primed SCX cartridge.
h) The SCX cartridge was purged with air.
i) The contents of the SCX cartridge were eluted into the reaction vial with 500 μL of the NaCl/HCl elution mixture followed by 0.5 mL air, using B. Braun Sterican needles to minimize leaching of metal ions from the needles. The contents of the reaction vial were briefly mixed and allowed to react at room temperature for 10 minutes.
j) 3 mL of phosphate buffer was added to the reaction vial. The contents of the reaction vial were withdrawn with a 10 mL syringe and passed through a 0.22 μm filter into a new sterile vial yielding the final product for injection. No post-purification of the product was performed as ⁶⁸Ga-NODAGA-GSAO was not significantly retained on C-18 cartridges and a suitable biocompatible post-purification cartridge/solvent system has not been identified. Despite this, the method described produced ⁶⁸Ga-NODAGA-GSAO of high radiochemical purity and specific activity, exceeding current release requirements for ⁶⁸Ga radiopharmaceuticals.
k) A sterile, closed radiolabelling system is used for the above procedure, as is preferred for preparation for human use and also for minimization of the risk of radioactive contamination to the operator and environment (FIG. 18). This may also be automated using a radiochemistry synthesis module.

### Purity of ⁶⁸Ga-NODAGA-GSAO

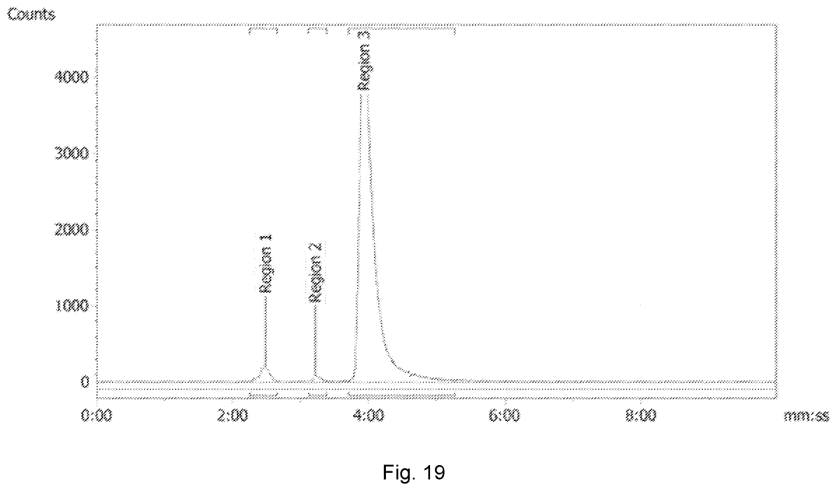

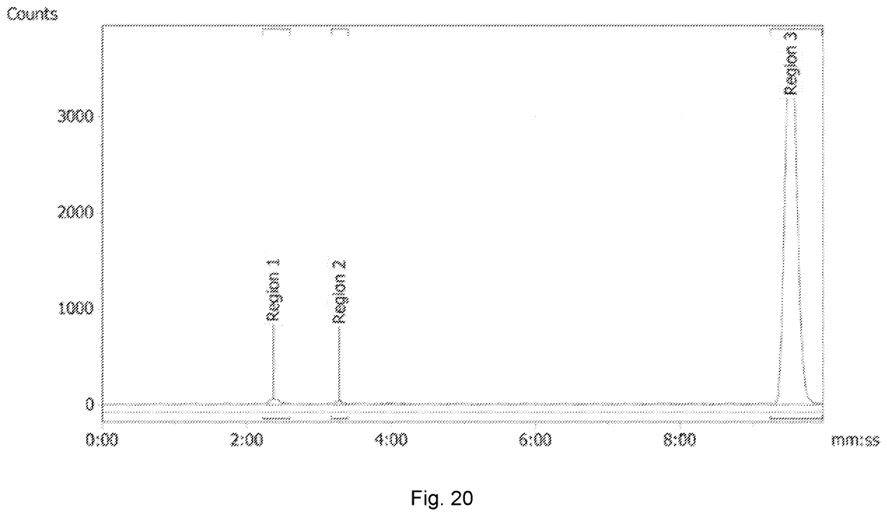

l) Radiochemical purity of ⁶⁸Ga-NODAGA-GSAO (approximately 100 μL sample of the final product obtained in step h) above) was assessed by HPLC system C at 9-9-60% mobile phase B (acetonitrile) in mobile phase A (0.1% TFA in ultrapure water) over 0-6-10 minutes using radiometric detection. The AUC of ⁶⁸Ga-NODAGA-GSAO peak over the sum of all radiometric peaks greater than three times background was used to determine radiochemical purity. Absorbance was also measured at 210 and 280 nm; however, the molar quantities were below the limits of reliable absorbance detection and were therefore not used for assessment of purity. ⁶⁸Ga-NODAGA-GSAO was eluted with a retention time of approximately 3 minutes and 55 seconds, as shown in the radiometric HPLC chromatogram of the final product in FIG. 19: region 1 is corresponds to ⁶⁸Ga, region 2 corresponds to oxidation products, and region 3 corresponds to ⁶⁸Ga-NODAGA-GSAO. The release criterion used for radiochemical purity of ⁶⁸Ga-NODAGA-GSAO in the final product was ≥91% (European Pharmacopeia (2016) 01/2013:2482 Gallium (68Ga) Edotreotide injection correct 8.6. European Pharmacopeia, 9^{th} edn, pp 1150-1152).
m) Further assessment of the radiochemical purity of ⁶⁸Ga-NODAGA-GSAO was performed by reacting 200 μL of the final product with 5 μL of DMP/DMSO solution at room temperature with occasional agitation for 10 minutes. Approximately 100 μL of this mixture was assessed by HPLC system C at 9-9-60% mobile phase B (acetonitrile) in mobile phase A (0.1% TFA in ultrapure water) over 0-6-10 minutes using radiometric detection. The DMP-⁶⁸Ga-NODAGA-GSAO peak (with a retention time of approximately 9 minutes and 30 seconds) over the sum of all radiometric peaks greater than three times background should be ≥91%; as DMP binds with very high affinity to the phenylarsonous moiety of ⁶⁸Ga-NODAGA-GSAO this will abolish the usual ⁶⁸Ga-NODAGA-GSAO peak with a retention time of approximately 3 minutes and 55 seconds and result in a new peak with a retention time of approximately 9 minutes and 30 seconds. This provides specific information about the radiochemical purity of the active GSAO and is able to distinguish between ⁶⁸Ga-NODAGA-GSAO and other products, such as oxidized degradation products of GSAO. However, this is not included in the required release criteria to minimise loss of product due to decay. The Radiometric HPLC chromatogram obtained is shown in FIG. 20: region 1 corresponds to unchelated ⁶⁸Ga, region 2 corresponds to oxidation products, and region 3 corresponds to DMP-⁶⁸Ga-NODAGA-GSAO.
n) Assessment of colloidal contaminants was performed by instant thin-layer chromatography developed in 0.9% NaCl. Colloidal contaminants remained at the origin while ⁶⁸Ga-NODAGA-GSAO had Rf>0.5. The release criterion used for colloid contaminants with total radioactivity with Rf≥0.5 was ≥90%.
o) Half-life was determined by a minimum of four measurements over 10 minutes performed on a dose calibrator. The release criterion used was a calculated half-life between 64 and 72 minutes (the half-life determination is required to confirm the absence of significant ⁶⁸Ge breakthrough).

### Sterility and Pyrogenicity Testing

p) Sterility and pyrogenicity were initially tested in an appropriately accredited laboratory on three serial syntheses to confirm that for the process sterility and pyrogenicity are within pharmacopoeia guidelines (European Pharmacopeia (2016) 01/2013:2482 Gallium ⁶⁸Ga Edotreotide injection correct 8.6. European Pharmacopeia, 9^{th} edn, pp 1150-1152). Subsequent random testing of preparations is performed at regular intervals.

### Example 7

### Pharmaceutical Formulation of ⁶⁸Ga-NODAGA-GSAO

A composition was prepared containing ingredients in the amounts listed in Table 5 below.

TABLE 5

| Ingredient Name | Quantity | Unit |
|---|---|---|
| Ascorbic acid | 4.4 | mg |
| Sodium | 95 | mg |
| Phosphate | 109 | mg |
| Acetate | 22 | mg |
| Chloride | 91 | mg |
| ⁶⁸Ga-NODAGA-GSAO | 200 | MBq |

### Example 8

### Biodistribution of ⁶⁸Ga-NODAGA-GSAO

Biodistribution was studied in ten healthy male rats (Lewis, Liverpool Hospital Animal Facility) aged 6-8 weeks. Five rats were administered with ⁶⁸Ga-NODAGA-GSAO. The rats were housed singly in a cage with impervious absorbent matting and at 1 hour post administration 5 rats were sacrificed by lethal carbon dioxide overdose. Immediately post mortem, blood was sampled via cardiac puncture. Two of the 5 rats were then imaged by PET CT (GE Discovery 710). The PET CT scan comprised a CT scan (80 kVp, 20 mA, helical mode, reconstructed slice thickness of 0.625 mm) followed by a PET scan (2 bed positions, 7.5 min/bed position, 256×256 reconstruction matrix, slice thickness 3.27 mm).

All of the rats were then dissected, organs sampled and weighed and counted in a gamma counter, and the cpm value converted to MBq using a known standard. The activity in the remaining carcass was measured in a dose calibrator.

Biodistribution studies were performed in a further 5 rats at two hours following ⁶⁸Ga-NODAGA GSAO administration.

Injected activity was corrected by measuring residual activity left in the syringe after injection in a dose calibrator. To correct for any dose extravasated at the injection site the tail was harvested and the activity in the tail was subtracted from the administered activity. All calculations were decayed corrected using the injection time as the reference.

Biodistribution was expressed as % ID/g and % ID/organ. % retained activity was the sum total of all activity in all individually harvested organs as well as the activity in the remaining carcass as a percentage of the injected dose. % recovered activity was the sum total of all activity in all individually harvested organs as well as the activity in the remaining carcass and excreted activity in the impervious matting as a percentage of the injected dose.

### Results

The rats weighed an average of 170 g (range 120-229 g, standard deviation 32.2 g). The average injected activity was 27.3 MBq (range 18.9-38.6 MBq, standard deviation 7.4 MBq).

For the 1 hour biodistribution group, the mean uptake time was 62.6 (range 60-65) minutes and for the 2 hour biodistribution group the mean uptake time was 122.2 (range 120-126) minutes.

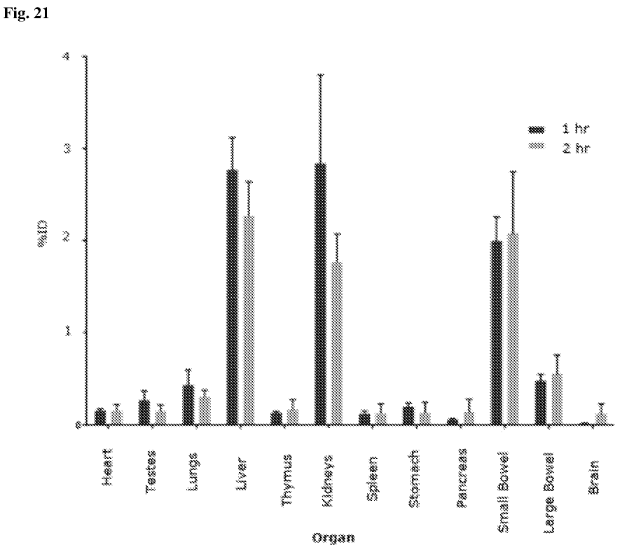

FIG. 21 shows the organ biodistribution of ⁶⁸Ga-NODAGA-GSAO (% ID/g) in healthy male rats at 1 and 2 hours post administration of ⁶⁸Ga-NODAGA-GSAO.

As seen in FIG. 21, the highest concentration of ⁶⁸Ga-NODAGA-GSAO is in the kidneys, and the organs with the greatest uptake of ⁶⁸Ga-NODAGA-GSAO are the kidneys, liver and small bowel. The high renal and hepatic uptake is consistent with renal excretion and hepatic metabolism while the small bowel uptake is likely to reflect uptake within dead and dying small bowel epithelial cells.

At 1 hour 32.4% (range 24.9-38.2%, SD 5.6%) of injected activity was retained and at 2 hours 21.4% (range 11.2-32.1%, SD 7.5%) of injected activity was retained within the animal. Overall mean total recovered activity at 1 hour was 84.9% (range 55.3-107.9%, SD 19.0%) and at 2 hours total recovered activity was 75.3% (range 50.0-120.9%, SD 27.2%) of injected activity.

### Imaging

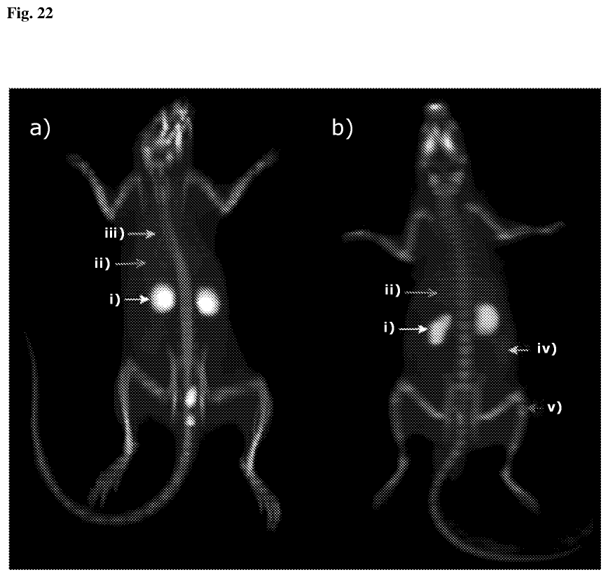

PET CT images demonstrated findings concordant with the quantitative biodistribution data. FIG. 22 shows the maximum intensity projections of ⁶⁸Ga-NODAGA-GSAO PET CT scans performed a) 1 hour and b) 2 hours following tracer (⁶⁸Ga-NODAGA-GSAO) administration. The images performed one hour after tracer administration demonstrate a high concentration of tracer in the kidneys (arrows i) in FIG. 22 a) and b)), with lower levels of uptake in the liver (arrows ii)). There is residual blood pool activity in the mediastinum (arrow iii)) similar to that of the liver. In the images performed 2 hours after administration (FIG. 22b) there is again a high concentration of tracer in the kidneys with lower levels of uptake in the liver. There is no longer visible blood pool activity in the mediastinum. In both the sets of images there is uptake in the small bowel (arrow iv)) and in the physes (arrow v)) likely due to specific uptake at sites of high physiological cell death.

### Example 9

### Radiation Dosimetry

The biodistribution data derived above was used to estimate human radiation dosimetry using the methods described by Stabin for a standard adult male (Stabin and Siegel 2003). The % ID/g for a given standard male organ was extrapolated from the rat biodistribution data using the following equation:

$${( \frac{\% ID}{Organ} )}_{human} = [ ( \frac{\% ID}{{g}_{organ}} ) \times {( {g}_{TB weight} )}_{animal} ] \times {( \frac{{g}_{organ}}{{g}_{TB weight}} )}_{human}$$

Mono-exponential clearance curves for each organ and total remaining tissues were fitted using tools in OLINDA/EXM software. Given the rapid excretion of ⁶⁸Ga-NODAGA-GSAO it was assumed that all excretion was via urine (i.e. urinary half clearance time was calculated using a mono-exponential fit and was assumed to be 1-00 total retained activity at each time point). For the voiding bladder model it was assumed that patients would void 1 hour following administration.

Whole body effective dose was estimated at 2.13E-02 mSv/MBq. Assuming an injected activity of 150 MBq this results in a whole body effective dose of 3.2 mSv, a dose lower than from a diagnostic CT scan of the abdomen and lower than FDG-PET CT. Estimated human individual organ doses are listed in Table 6 below (ULI=upper large intestine, LLI=lower large intestine).

TABLE 6

| Target Organ | Alpha | Beta | Photon | Total | ED Cont. |
|---|---|---|---|---|---|
| Adrenals | 0.00E000 | 2.13E−03 | 1.89E−03 | 4.02E−03 | 2.01E−05 |
| Brain | 0.00E000 | 8.15E−04 | 5.73E−04 | 1.39E−03 | 6.94E−06 |
| Breasts | 00.00E000 | 2.13E−03 | 8.94E−04 | 3.02E−03 | 1.51E−04 |
| Gallbladder | 0.00E000 | 2.13E−03 | 2.15E−03 | 4.28E−03 | 0.00E000 |
| Wall | | | | | |
| LLI Wall | 0.00E000 | 2.13E−03 | 5.09E−03 | 7.23E−03 | 8.67E−04 |
| Small Intestine | 0.00E000 | 5.18E−03 | 3.03E−03 | 8.21E−03 | 4.10E−05 |
| Stomach Wall | 0.00E000 | 2.61E−03 | 1.61E−03 | 4.22E−03 | 5.06E−04 |
| ULI Wall | 0.00E000 | 2.53E−03 | 2.63E−03 | 5.16E−03 | 2.58E−05 |
| Heart Wall | 0.00E000 | 3.09E−03 | 1.55E−03 | 4.65E−03 | 0.00E000 |
| Kidneys | 0.00E000 | 2.27E−02 | 3.56E−03 | 2.62E−02 | 1.31E−04 |
| Liver | 0.00E000 | 4.76E−03 | 2.04E−03 | 6.80E−03 | 3.40E−04 |
| Lungs | 0.00E000 | 5.42E−03 | 1.38E−03 | 6.80E−03 | 8.16E−04 |
| Muscle | 0.00E000 | 2.13E−03 | 2.04E−03 | 4.18E−03 | 2.09E−05 |
| Ovaries | 0.00E000 | 2.13E−03 | 4.89E−03 | 7.02E−03 | 1.40E−03 |
| Pancreas | 0.00E000 | 3.87E−03 | 2.03E−03 | 5.90E−03 | 2.95E−05 |
| Red Marrow | 0.00E000 | 1.49E−03 | 1.89E−03 | 3.38E−03 | 4.05E−04 |
| Osteogenic | 0.00E000 | 3.31E−03 | 1.67E−03 | 4.98E−03 | 4.98E−05 |
| Cells | | | | | |
| Skin | 0.00E000 | 2.13E−03 | 1.06E−03 | 3.19E−03 | 3.19E−05 |
| Spleen | 0.00E000 | 4.05E−03 | 1.86E−03 | 5.91E−03 | 2.95E−05 |
| Testes | 0.00E000 | 2.21E−03 | 3.49E−03 | 5.69E−03 | 0.00E000 |
| Thymus | 0.00E000 | 4.48E−03 | 1.36E−03 | 5.84E−03 | 2.92E−05 |
| Thyroid | 0.00E000 | 2.13E−03 | 1.15E−03 | 3.28E−03 | 1.64E−04 |
| Urinary | 0.00E000 | 2.84E−01 | 3.92E−02 | 3.24E−01 | 1.62E−02 |
| Bladder | | | | | |
| Wall | | | | | |
| Uterus | 0.00E000 | 2.13E−03 | 9.42E−03 | 1.15E−02 | 5.77E−05 |
| Total Body | 0.00E000 | 2.68E−03 | 1.93E−03 | 4.61E−03 | 0.00E000 |

### Discussion

As shown in the above-described experiments, ⁶⁸Ga-NODAGA-GSAO has advantageous imaging characteristics, with relatively little interference from physiologic renal and hepatic activity. In addition, the rapid clearance suggests that imaging between 1 and 2 hours post injection is feasible and thus well suited to using ⁶⁸Ga (clinically for ⁶⁸Ga-based somatostatin receptor expression imaging, imaging is performed at 45-90 minutes following injection). Of note from the ⁶⁸Ga-NODAGA-GSAO PET/CT images (FIG. 22) is the visualisation of uptake within small and large bowel and also in the physes of the long bones, which may represent uptake in areas of high rates of physiologic cell death. The imaging appearances are confirmed by the measured distribution, and in contrast to some other organs (especially the liver and kidneys) uptake is higher at the 2 hour time point then at one hour post-injection, suggesting that the uptake in bowel may represent specific binding rather than non-specific tracer diffusion.

The estimated human radiation dosimetry is favourable, with an estimated total body effective dose of 0.021 mSv/MBq which, assuming a standard injected dose of 150 MBq, would deliver a total dose whole body effective dose of 3.2 mSv. The dose limiting organ is the urinary bladder wall with a dose of 0.32 mSv/MBq.

These combined results suggest that ⁶⁸Ga-NODAGA-GSAO may be a promising agent for in vivo imaging of dead and dying cells and first in human studies are warranted.

### Example 10

### Human Studies

The following patients were administered between 200 and 207 MBq 200 MBq of ⁶⁸Ga-NODAGA-GSAO:
1. 66 year old male patient with squamous cell carcinoma of the oesophagus
2. 73 year old female with metastatic ovarian carcinoma
3. 66 year old male with metastatic cutaneous squamous cell carcinoma
4. 81 year old female with invasive ductal breast carcinoma.

All subjects tolerated the study well with no related or unrelated serious adverse events or adverse events. There were no significant changes in any clinical, laboratory or electrocardiographic parameters.

### Biodistribution

The biodistribution data demonstrates prompt intravascular distribution of ⁶⁸Ga-NODAGA-GSAO with rapid initial clearance, followed by a second slower phase of clearance from the blood pool. There is rapid renal uptake and excretion.

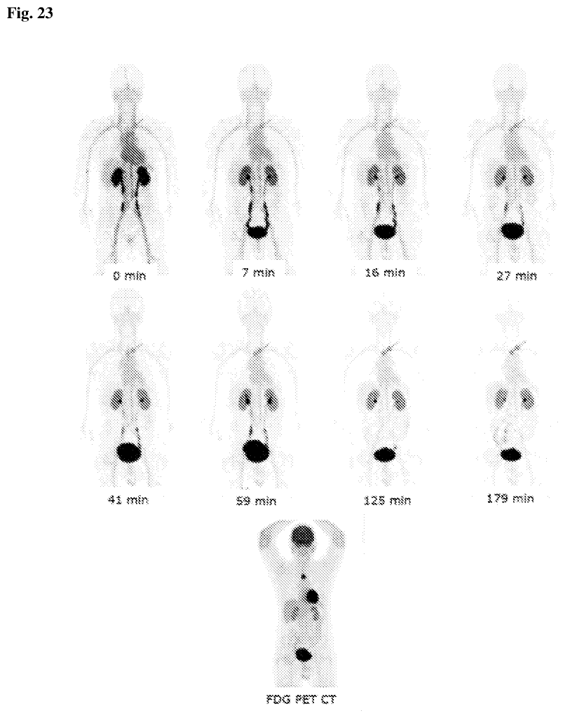

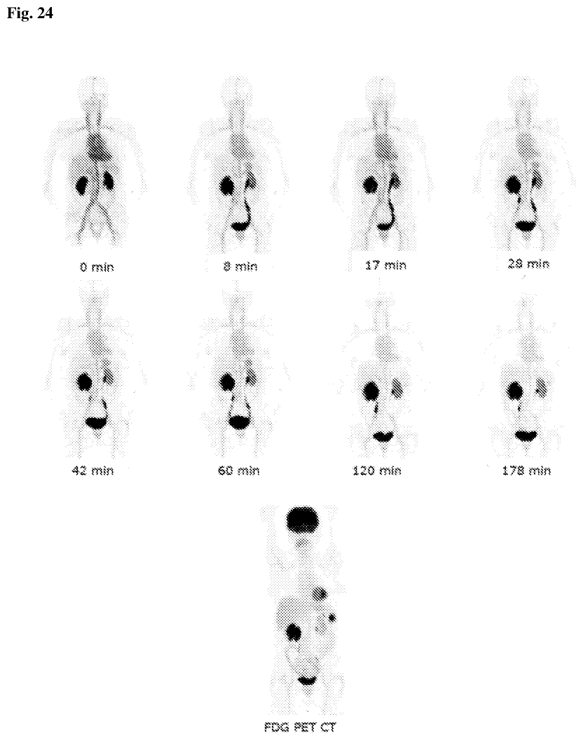

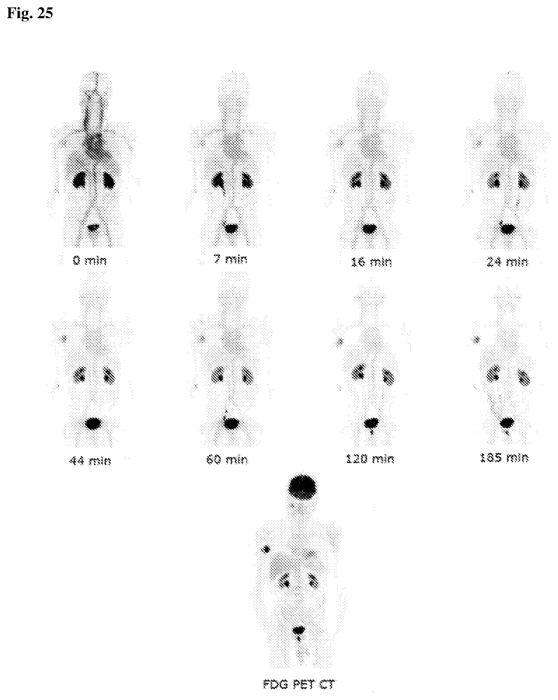

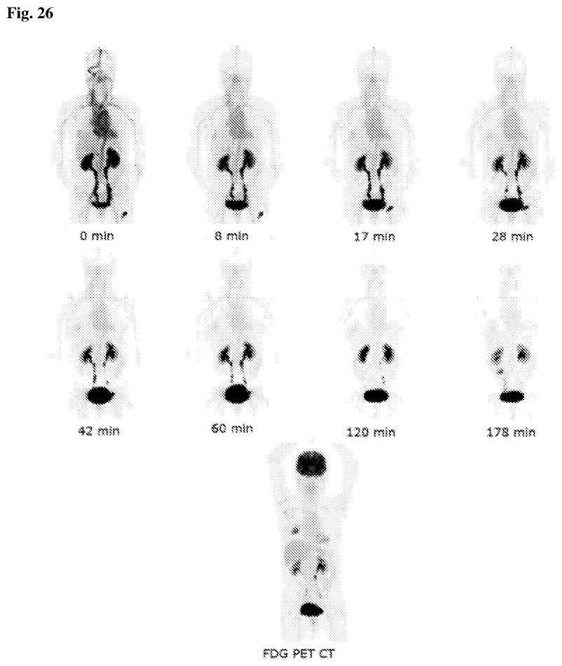

For patient 1), the % injected dose (% ID) excreted in urine by 2 hours averaged 30% (range 19-38%) and by 3 hours averaged 48% (range 21-71%). Imaging findings from this subject are shown in FIG. 23, which shows anterior maximum intensity projections of ⁶⁸Ga-NODAGA-GSAO PET at 8 time points; anterior maximum projection of the FDG PET is shown underneath for comparison. The location of the tumour is arrowed at each time point. Low levels of tracer uptake are seen in the remaining organs which gradually declines over time (apart from the testis and large bowel). No hepatobiliary excretion is evident. There is almost absent activity within the brain, suggesting that it does not cross the blood brain barrier to any extent. Imaging finding from patients 2-4 are similarly shown in FIG. 24 (patient 2) FIG. 25 (patient 3), FIG. 26 (patient 4).

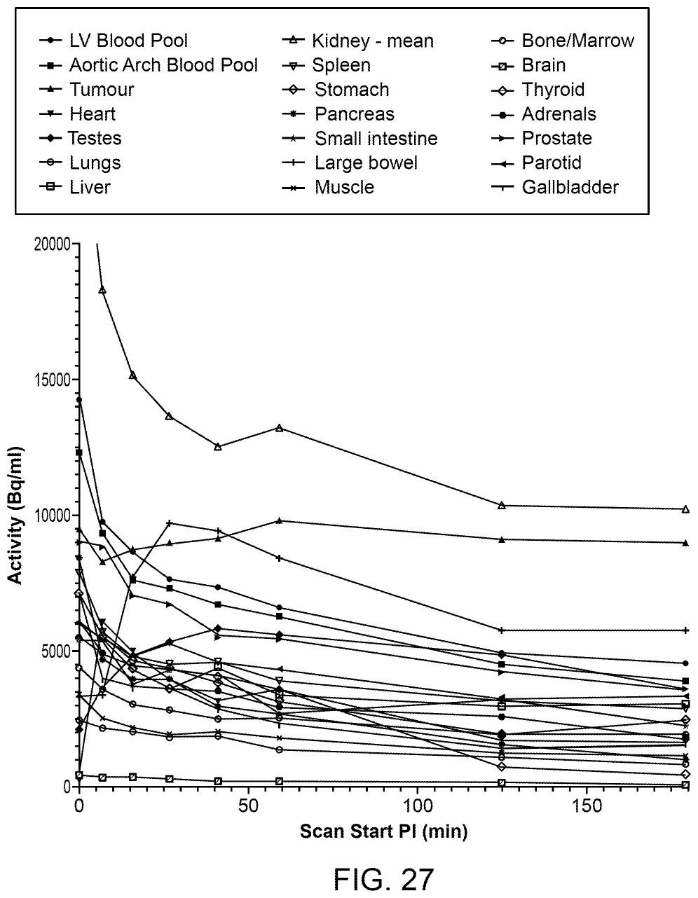

FIG. 27 shows biodistribution of ⁶⁸Ga-NODAGA-GSAO in normal organs over time in patient 1. In blood there is an initial rapid decrease in concentration, followed by a second slower phase of clearance. Most of the organs demonstrate an early peak followed by a gradual decline, similar to the second phase of blood clearance, except for the large bowel and testes which demonstrate an initial increase in concentration up to approximately 40 minutes following administration and then a slow decline. This may be due to higher physiologic rates of cell death in these two organs. Note that the urinary bladder wall was evaluated separately.

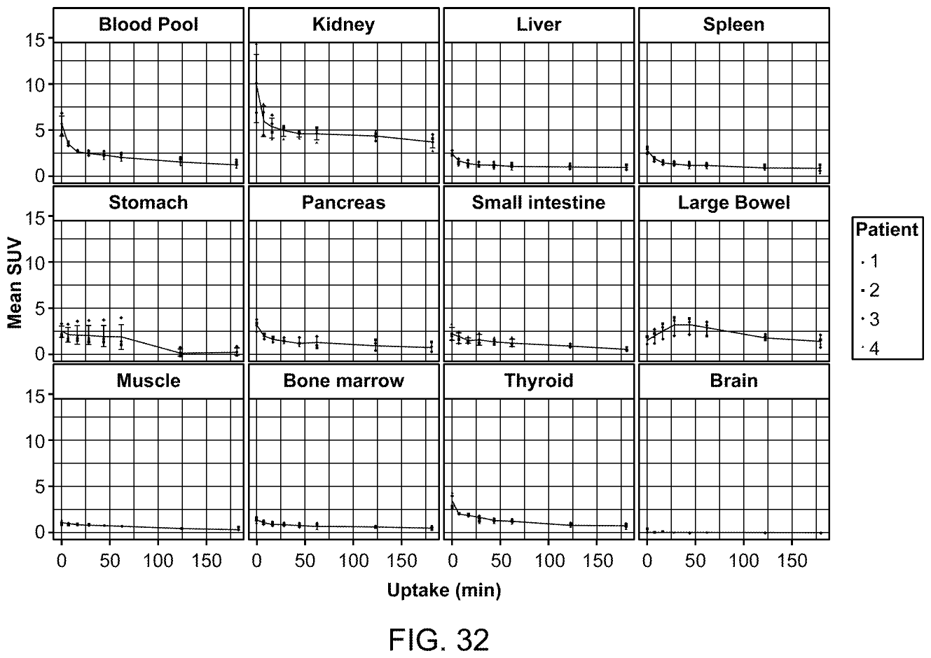

Patterns of biodistribution in organs and tissues were consistent across subjects 1-4 (as shown in FIG. 32). All demonstrated rapid distribution of ⁶⁸Ga NODAGA GSAO through the blood pool following injection, with rapid renal uptake and excretion. At 1 h post injection, the kidneys had the highest concentration of ⁶⁸Ga NODAGA GSAO (4.85±0.70; mean SUV SD, SUV=Standard Uptake Value) with relatively low levels of ⁶⁸Ga NODAGA GSAO in the other tissues and organs, which cleared over time. The large bowel has the next highest concentration of ⁶⁸Ga NODAGA GSAO (3.00±0.62), followed by the blood pool (2.31±0.37) and stomach (2.05±1.34).

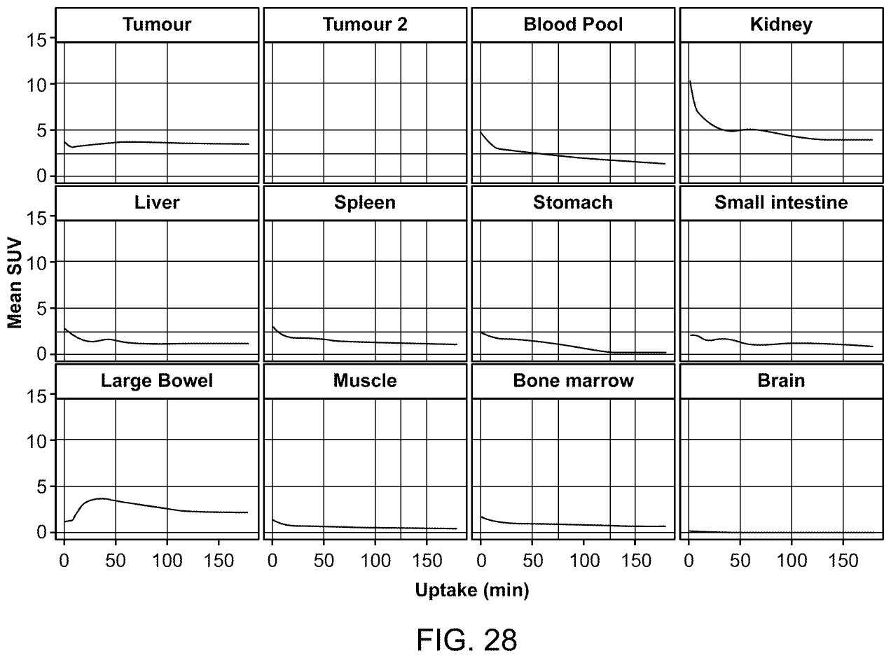

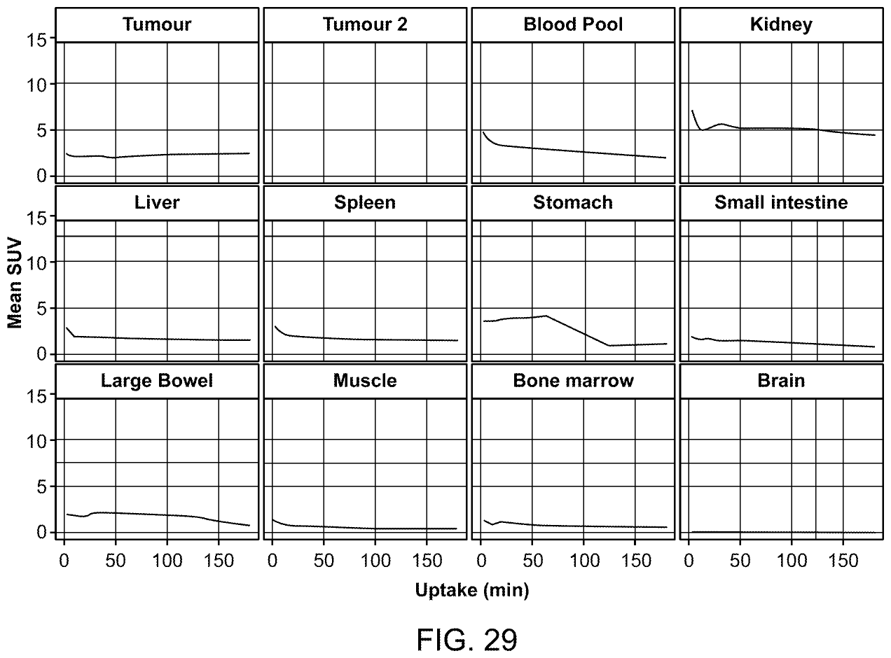

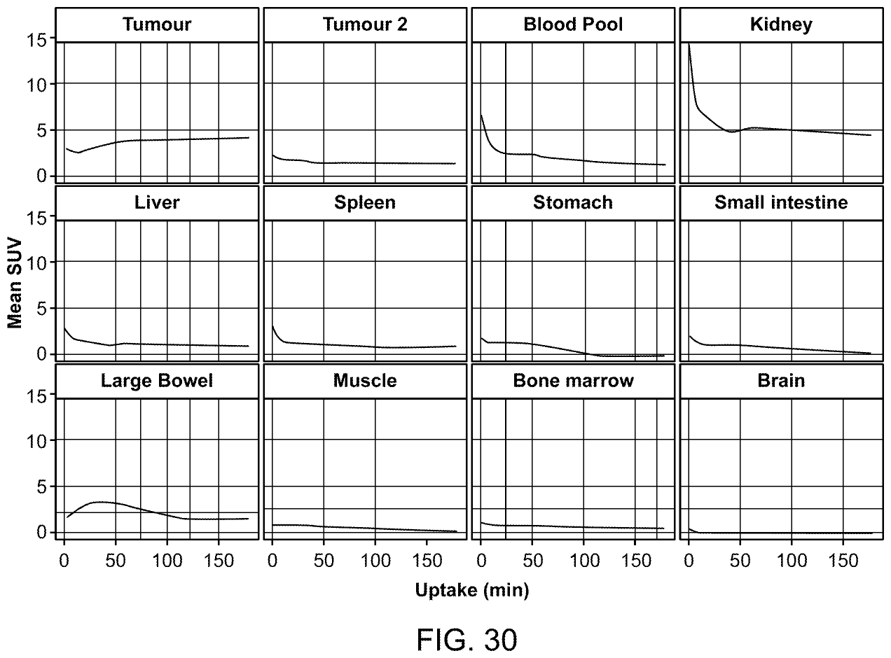

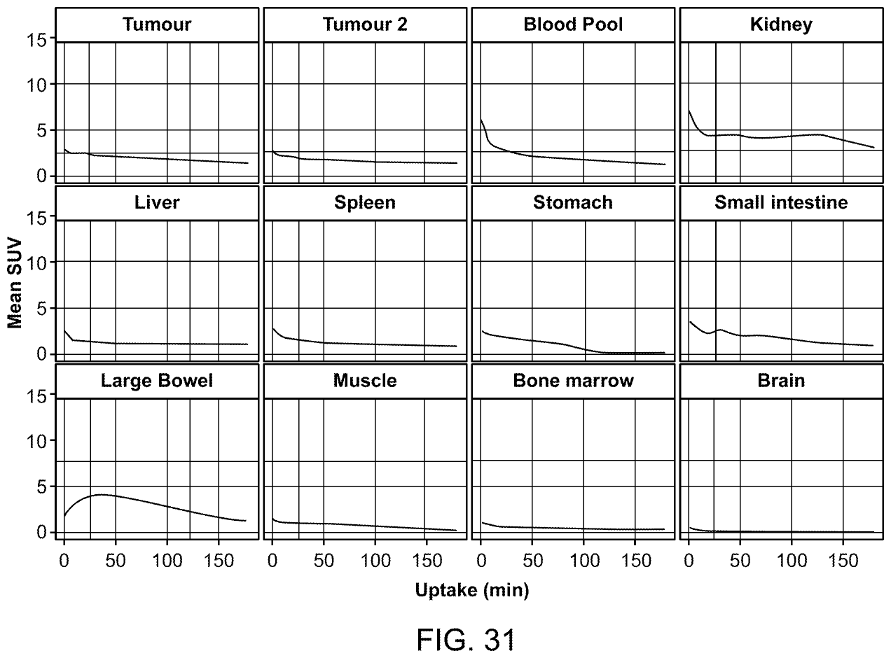

FIGS. 28-31 show biodistribution of ⁶⁸Ga NODAGA GSAO in selected normal tissues and tumour for patients 1-4 respectively. Note that Tumour 2 is only applicable in patients 3 and 4, so is blank in FIGS. 28 and 29. FIG. 32 shows the biodistribution in selected normal tissues (mean SUV±SD) of subjects 1-4.

### Radiation Dosimetry

The whole-body effective dose was estimated by drawing representative spherical volumes of interest within the organs, estimating the % ID/g for each organ and then calculating the % ID/organ using the organ weights from a standard adult phantom.

The effective whole-body dose from ⁶⁸Ga NODAGA GSAO for subjects 1-4 ranged from 2.16×10⁻² to 3.38×10⁻² mSv/MBq, giving an estimated effective whole-body dose ranging from 13.5-15.9 mSv for the protocol used in the first in human study. Detailed organ dosimetry for ⁶⁸Ga NODAGA GSAO is shown for the four subjects (tables 7-10). In all cases, the urinary bladder was the dose limiting organ. For subsequent human studies, fewer time points will be required, reducing the need for low dose CTs which will reduce the overall radiation dose. The dose is at level that is comparable to many routine medical imaging procedures using ionising radiation including x-ray computed tomography (CT), SPECT/CT and PET/CT scans.

For subjects 1-4, radiation dosimetry was calculated using Olinda/EXM based on the organ biodistribution discussed above. Urinary excretion was modelled based on measurement of activity in collected urine samples and urinary volume was measured from the images.

Tables 7-10 show the estimate for radiation dosimetry for subjects 1-4 of 200 MBq of ⁶⁸Ga NODAGA GSAO for individual organs and for the whole body in mSv/MBq (EDE cont.=effective dose equivalent contribution, ED Cont.=effective dose contribution). The estimated whole-body dose from the one (1) low dose CT and two (2) ultra-low dose CTs was 9.2 mSv.

Table 7 shows the estimate for radiation dosimetry for subject 1. The overall estimated radiation dose to subject 1 was 14.5 mSv.

TABLE 7

| Target Organ | Alpha | Beta | Photon | Total | EDE Cont. | ED Cont. |
|---|---|---|---|---|---|---|
| Adrenals | 0.00E+00 | 9.68E−03 | 4.52E−03 | 1.42E−02 | 2.68E−04 | 7.09E−05 |
| Brain | 0.00E+00 | 6.82E−04 | 1.14E−03 | 1.82E−03 | 0.00E+00 | 9.10E−06 |
| Breasts | 0.00E+00 | 5.07E−03 | 2.11E−03 | 7.16E−03 | 1.08E−03 | 3.59E−04 |
| Gallbladder Wall | 0.00E+00 | 3.48E−03 | 4.59E−03 | 8.07E−03 | 0.00E+00 | 0.00E+00 |
| LLI Wall | 0.00E+00 | 1.29E−02 | 7.31E−03 | 2.02E−02 | 1.03E−03 | 2.43E−03 |
| Small Intestine | 0.00E+00 | 9.17E−03 | 5.21E−03 | 1.44E−02 | 0.00E+00 | 7.19E−05 |
| Stomach Wall | 0.00E+00 | 6.13E−03 | 3.93E−03 | 1.01E−02 | 0.00E+00 | 1.21E−03 |
| ULI Wall | 0.00E+00 | 7.04E−03 | 5.20E−03 | 1.22E−02 | 0.00E+00 | 6.12E−05 |
| Heart Wall | 0.00E+00 | 6.65E−03 | 3.57E−03 | 1.02E−02 | 0.00E+00 | 0.00E+00 |
| Kidneys | 0.00E+00 | 4.32E−02 | 7.22E−03 | 5.04E−02 | 3.03E−03 | 2.52E−04 |
| Liver | 0.00E+00 | 1.27E−02 | 4.84E−03 | 1.76E−02 | 5.09E−04 | 8.78E−04 |
| Lungs | 0.00E+00 | 5.19E−03 | 2.93E−03 | 8.12E−03 | 9.74E−04 | 9.74E−04 |
| Muscle | 0.00E+00 | 6.19E−03 | 3.51E−03 | 9.71E−03 | 0.00E+00 | 4.85E−05 |
| Ovaries | 0.00E+00 | 3.15E−03 | 7.00E−03 | 1.01E−02 | 1.50E−03 | 1.20E−03 |
| Pancreas | 0.00E+00 | 1.20E−02 | 4.90E−03 | 1.69E−02 | 8.14E−04 | 8.46E−05 |
| Red Marrow | 0.00E+00 | 5.39E−03 | 3.70E−03 | 9.08E−03 | 1.09E−03 | 1.09E−03 |
| Osteogenic Cells | 0.00E+00 | 7.79E−03 | 3.47E−03 | 1.12E−02 | 3.38E−04 | 1.12E−04 |
| Skin | 0.00E+00 | 3.15E−03 | 2.03E−03 | 5.17E−03 | 0.00E+00 | 5.17E−05 |
| Spleen | 0.00E+00 | 1.27E−02 | 4.57E−03 | 1.73E−02 | 5.68E−04 | 8.65E−05 |
| Testes | 0.00E+00 | 1.37E−02 | 4.58E−03 | 1.83E−02 | 4.56E−03 | 3.65E−03 |
| Thymus | 0.00E+00 | 3.15E−03 | 2.96E−03 | 6.11E−03 | 0.00E+00 | 3.06E−05 |
| Thyroid | 0.00E+00 | 1.12E−02 | 2.86E−03 | 1.41E−02 | 4.23E−04 | 7.05E−04 |
| Urinary Bladder Wall | 0.00E+00 | 2.52E−01 | 4.05E−02 | 2.93E−01 | 1.76E−02 | 1.46E−02 |
| Uterus | 0.00E+00 | 8.21E−03 | 1.06E−02 | 1.89E−02 | 5.83E−04 | 9.43E−05 |
| Total Body | 0.00E+00 | 6.33E−03 | 3.44E−03 | 9.78E−03 | 0.00E+00 | 0.00E+00 |
| Effective Dose | | | | | | 2.63E−02 |

Table 8 shows the estimate for radiation dosimetry for subject 2. The overall estimated radiation dose to subject 2 was 13.9 mSv.

TABLE 8

| Target Organ | Alpha | Beta | Photon | Total | EDE Cont | ED Cont |
|---|---|---|---|---|---|---|
| Adrenals | 0.00E+00 | 9.53E−03 | 5.24E−03 | 1.48E−02 | 0.00E+00 | 7.38E−05 |
| Brain | 0.00E+00 | 6.40E−04 | 1.46E−03 | 2.10E−03 | 0.00E+00 | 1.05E−05 |
| Breasts | 0.00E+00 | 9.48E−03 | 2.76E−03 | 1.22E−02 | 1.84E−03 | 6.12E−04 |
| Gallbladder Wall | 0.00E+00 | 4.69E−03 | 5.27E−03 | 9.96E−03 | 0.00E+00 | 0.00E+00 |
| LLI Wall | 0.00E+00 | 1.37E−02 | 7.16E−03 | 2.08E−02 | 1.25E−03 | 2.50E−03 |
| Small Intestine | 0.00E+00 | 1.27E−02 | 5.57E−03 | 1.83E−02 | 0.00E+00 | 9.14E−05 |
| Stomach Wall | 0.00E+00 | 1.15E−02 | 4.97E−03 | 1.65E−02 | 0.00E+00 | 1.98E−03 |
| ULI Wall | 0.00E+00 | 1.19E−02 | 6.02E−03 | 1.79E−02 | 0.00E+00 | 8.97E−05 |
| Heart Wall | 0.00E+00 | 8.27E−03 | 4.29E−03 | 1.26E−02 | 0.00E+00 | 0.00E+00 |
| Kidneys | 0.00E+00 | 4.21E−02 | 7.59E−03 | 4.97E−02 | 2.98E−03 | 2.48E−04 |
| Liver | 0.00E+00 | 1.45E−02 | 5.51E−03 | 2.00E−02 | 0.00E+00 | 1.00E−03 |
| Lungs | 0.00E+00 | 5.73E−03 | 3.54E−03 | 9.27E−03 | 1.11E−03 | 1.11E−03 |
| Muscle | 0.00E+00 | 6.08E−03 | 3.83E−03 | 9.91E−03 | 0.00E+00 | 4.95E−05 |
| Ovaries | 0.00E+00 | 4.69E−03 | 6.95E−03 | 1.16E−02 | 2.91E−03 | 2.33E−03 |
| Pancreas | 0.00E+00 | 1.62E−02 | 5.90E−03 | 2.21E−02 | 1.32E−03 | 1.10E−04 |
| Red Marrow | 0.00E+00 | 6.61E−03 | 4.24E−03 | 1.08E−02 | 1.30E−03 | 1.30E−03 |
| Osteogenic Cells | 0.00E+00 | 1.14E−02 | 4.14E−03 | 1.55E−02 | 4.65E−04 | 1.55E−04 |
| Skin | 0.00E+00 | 4.69E−03 | 2.32E−03 | 7.01E−03 | 0.00E+00 | 7.01E−05 |
| Spleen | 0.00E+00 | 1.48E−02 | 5.34E−03 | 2.02E−02 | 1.21E−03 | 1.01E−04 |
| Thymus | 0.00E+00 | 4.69E−03 | 3.66E−03 | 8.36E−03 | 0.00E+00 | 4.18E−05 |
| Thyroid | 0.00E+00 | 1.19E−02 | 3.24E−03 | 1.52E−02 | 4.55E−04 | 7.59E−04 |
| Urinary Bladder Wall | 0.00E+00 | 1.84E−01 | 3.21E−02 | 2.16E−01 | 1.30E−02 | 1.08E−02 |
| Uterus | 0.00E+00 | 4.69E−03 | 8.98E−03 | 1.37E−02 | 0.00E+00 | 6.84E−05 |
| Total Body | 0.00E+00 | 7.76E−03 | 3.84E−03 | 1.16E−02 | 0.00E+00 | 0.00E+00 |
| Effective Dose | | | | | | 2.35E−02 |

Table 9 shows the estimate for radiation dosimetry for subject 3. The overall estimated radiation dose to subject 3 was 13.5 mSv.

TABLE 9

| Target Organ | | | | | EDE Cont. | ED Cont. |
|---|---|---|---|---|---|---|
| Adrenals | 0.00E+00 | 5.97E−03 | 3.71E−03 | 9.68E−03 | 0.00E+00 | 4.84E−05 |
| Brain | 0.00E+00 | 6.13E−04 | 8.65E−04 | 1.48E−03 | 0.00E+00 | 7.39E−06 |
| Breasts | 0.00E+00 | 2.02E−03 | 1.59E−03 | 3.61E−03 | 5.41E−04 | 1.80E−04 |
| Gallbladder Wall | 0.00E+00 | 2.02E−03 | 4.07E−03 | 6.09E−03 | 0.00E+00 | 0.00E+00 |
| LLI Wall | 0.00E+00 | 1.44E−02 | 5.96E−03 | 2.03E−02 | 1.22E−03 | 2.44E−03 |
| Small Intestine | 0.00E+00 | 8.73E−03 | 4.29E−03 | 1.30E−02 | 0.00E+00 | 6.51E−05 |
| Stomach Wall | 0.00E+00 | 4.31E−03 | 3.12E−03 | 7.43E−03 | 0.00E+00 | 8.91E−04 |
| ULI Wall | 0.00E+00 | 2.02E−03 | 4.00E−03 | 6.02E−03 | 0.00E+00 | 3.01E−05 |
| Heart Wall | 0.00E+00 | 4.65E−03 | 2.82E−03 | 7.48E−03 | 0.00E+00 | 0.00E+00 |
| Kidneys | 0.00E+00 | 4.45E−02 | 6.83E−03 | 5.13E−02 | 3.08E−03 | 2.57E−04 |
| Liver | 0.00E+00 | 1.20E−02 | 4.36E−03 | 1.64E−02 | 9.84E−04 | 8.20E−04 |
| Lungs | 0.00E+00 | 5.16E−03 | 2.37E−03 | 7.53E−03 | 9.04E−04 | 9.04E−04 |
| Muscle | 0.00E+00 | 5.15E−03 | 2.83E−03 | 7.98E−03 | 0.00E+00 | 3.99E−05 |
| Ovaries | 0.00E+00 | 2.02E−03 | 5.52E−03 | 7.54E−03 | 0.00E+00 | 0.00E+00 |
| Pancreas | 0.00E+00 | 1.13E−02 | 4.18E−03 | 1.54E−02 | 9.27E−04 | 7.72E−05 |
| Red Marrow | 0.00E+00 | 4.29E−03 | 2.96E−03 | 7.25E−03 | 8.70E−04 | 8.70E−04 |
| Osteogenic Cells | 0.00E+00 | 4.95E−03 | 2.74E−03 | 7.69E−03 | 2.31E−04 | 7.69E−05 |
| Skin | 0.00E+00 | 2.02E−03 | 1.63E−03 | 3.66E−03 | 0.00E+00 | 3.66E−05 |
| Spleen | 0.00E+00 | 1.09E−02 | 3.83E−03 | 1.47E−02 | 0.00E+00 | 7.35E−05 |
| Testes | 0.00E+00 | 1.22E−02 | 4.18E−03 | 1.64E−02 | 4.09E−03 | 3.27E−03 |
| Thymus | 0.00E+00 | 2.02E−03 | 2.30E−03 | 4.32E−03 | 0.00E+00 | 2.16E−05 |
| Thyroid | 0.00E+00 | 8.46E−03 | 2.38E−03 | 1.08E−02 | 3.25E−04 | 5.42E−04 |
| Urinary Bladder Wall | 0.00E+00 | 1.91E−01 | 2.79E−02 | 2.19E−01 | 1.31E−02 | 1.09E−02 |
| Uterus | 0.00E+00 | 2.02E−03 | 8.36E−03 | 1.04E−02 | 0.00E+00 | 5.19E−05 |
| Total Body | 0.00E+00 | 4.94E−03 | 2.77E−03 | 7.71E−03 | 0.00E+00 | 0.00E+00 |
| Effective Dose | | | | | | 2.16E−02 |

Table 10 shows the estimate for radiation dosimetry for subject 4. The overall estimated radiation dose to subject 4 was 15.9 mSv.

TABLE 10
Photon Total

| Target Organ | Alpha | Beta | Photon | Total | EDE Cont. | ED Cont. |
|---|---|---|---|---|---|---|
| Adrenals | 0.00E+00 | 1.30E−02 | 4.86E−03 | 1.79E−02 | 1.07E−03 | 8.93E−05 |
| Brain | 0.00E+00 | 7.79E−04 | 1.23E−03 | 2.01E−03 | 0.00E+00 | 1.00E−05 |
| Breasts | 0.00E+00 | 6.43E−03 | 2.25E−03 | 8.68E−03 | 1.30E−03 | 4.34E−04 |
| Gallbladder Wall | 0.00E+00 | 4.09E−03 | 4.46E−03 | 8.56E−03 | 0.00E+00 | 0.00E+00 |
| LLI Wall | 0.00E+00 | 3.53E−03 | 8.79E−03 | 1.23E−02 | 0.00E+00 | 1.48E−03 |
| Small Intestine | 0.00E+00 | 3.53E−03 | 5.67E−03 | 9.20E−03 | 0.00E+00 | 4.60E−05 |
| Stomach Wall | 0.00E+00 | 3.53E−03 | 4.01E−03 | 7.54E−03 | 0.00E+00 | 9.05E−04 |
| ULI Wall | 0.00E+00 | 3.53E−03 | 5.50E−03 | 9.03E−03 | 0.00E+00 | 4.52E−05 |
| Heart Wall | 0.00E+00 | 7.57E−03 | 3.84E−03 | 1.14E−02 | 0.00E+00 | 0.00E+00 |
| Kidneys | 0.00E+00 | 4.30E−02 | 7.33E−03 | 5.03E−02 | 3.02E−03 | 2.52E−04 |
| Liver | 0.00E+00 | 1.16E−02 | 4.71E−03 | 1.63E−02 | 0.00E+00 | 8.13E−04 |
| Lungs | 0.00E+00 | 4.82E−03 | 3.15E−03 | 7.97E−03 | 9.57E−04 | 9.57E−04 |
| Muscle | 0.00E+00 | 7.68E−03 | 4.07E−03 | 1.18E−02 | 0.00E+00 | 5.88E−05 |
| Ovaries | 0.00E+00 | 3.53E−03 | 8.79E−03 | 1.23E−02 | 3.08E−03 | 2.46E−03 |
| Pancreas | 0.00E+00 | 1.18E−02 | 5.04E−03 | 1.69E−02 | 1.01E−03 | 8.44E−05 |
| Red Marrow | 0.00E+00 | 5.25E−03 | 4.13E−03 | 9.38E−03 | 1.13E−03 | 1.13E−03 |
| Osteogenic Cells | 0.00E+00 | 8.81E−03 | 3.80E−03 | 1.26E−02 | 3.78E−04 | 1.26E−04 |
| Skin | 0.00E+00 | 3.53E−03 | 2.26E−03 | 5.79E−03 | 0.00E+00 | 5.79E−05 |
| Spleen | 0.00E+00 | 1.18E−02 | 4.70E−03 | 1.65E−02 | 0.00E+00 | 8.27E−05 |
| Thymus | 0.00E+00 | 3.53E−03 | 3.23E−03 | 6.76E−03 | 0.00E+00 | 3.38E−05 |
| Thyroid | 0.00E+00 | 1.34E−02 | 3.00E−03 | 1.64E−02 | 4.91E−04 | 8.18E−04 |
| Urinary Bladder Wall | 0.00E+00 | 4.06E−01 | 6.85E−02 | 4.74E−01 | 2.85E−02 | 2.37E−02 |
| Uterus | 0.00E+00 | 2.38E−02 | 1.51E−02 | 3.89E−02 | 2.33E−03 | 1.95E−04 |
| Total Body | 0.00E+00 | 6.96E−03 | 3.92E−03 | 1.09E−02 | 0.00E+00 | 0.00E+00 |
| Effective Dose | | | | | | 3.38E−02 |

### Tumour Uptake

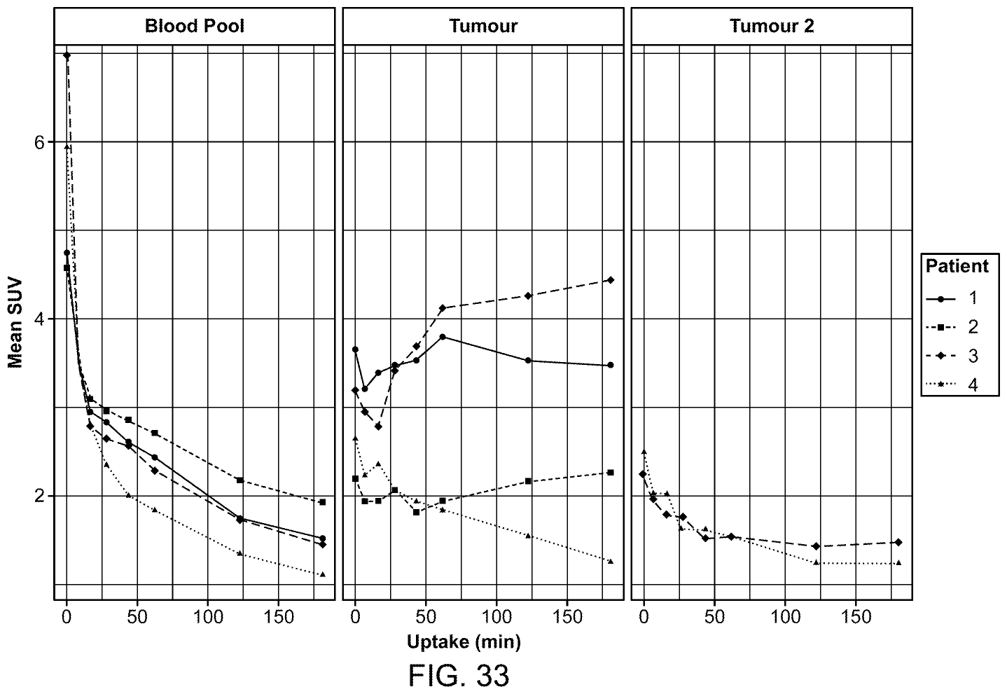

FIG. 33 shows blood pool activity and uptake of ⁶⁸Ga NODAGA GSAO into tumour deposits in subjects 1-4 (note: in patients 3 and 4, there are two tumour deposits, and these have been analysed separately). Whilst blood pool and clearance are reproducible, tumour uptake and clearance vary by tumour type.

Across subjects 1-4, tumour uptake was variable depending on tumour histology, with high levels of uptake seen in squamous cell carcinoma of the oesophagus (SUVmean 3.8) and metastatic cutaneous squamous cell carcinoma (SUVmean 4.1) and lower uptake seen in metastatic ovarian carcinoma (SUVmean 1.9) and breast carcinoma (SUVmean 1.8). Note that in subjects 3 and 4 there were two tumour deposits and these have been analysed separately. It is not unexpected that different tumour histology will have differing rates of de novo cell death. To confirm this, histological correlation of tumour cell death with tumour uptake of ⁶⁸Ga NODAGA GSAO was performed on two tumour deposits in patient 3 (one with high uptake of ⁶⁸Ga NODAGA GSAO SUVmean 4.1 in the right axilla and the other with low uptake of ⁶⁸Ga NODAGA GSAO SUVmean 2.7 in the right upper anterior cervical triangle) (FIG. 34).

Dissected tumours were fixed in formalin, embedded in paraffin and 4 μm thick sections were cut. Adjacent sections were stained for apoptotic cells using TUNEL (Abcam, Cat #206386) or morphology using haematoxylin and eosin. For TUNEL staining, sections were deparaffinized in xylene, rehydrated in decreasing concentrations of ethanol and permeabilized with Proteinase K for 20 min at room temperature. The endogenous peroxidase activity was quenched with 3% H₂O₂ for 5 min. Apoptotic cells were labelled with biotinylated terminal deoxynucleotidyl transferase at 37° C. in a humidified chamber for 2 h followed by a 30 min incubation with streptavidin-HRP conjugate. HRP-positive cells were developed using diaminobenzidine and sections counterstained with methyl green (Sigma). Whole sections were imaged using PowerMosaic scanning at 10× magnification on a Leica DM6000D microscope.

FIG. 34 shows anterior maximum projection intensity images of FDG-PET (FIG. 34A) performed 60 min after administration of 256 MBq of FDG (Fluorodeoxyglucose), and CDI-PET (FIG. 34B) performed 60 min after administration of 205 MBq of CDI (⁶⁸Ga NODAGA GSAO) in a 66 year old male with metastatic cutaneous squamous cell carcinoma (patient 3). The FDG-PET demonstrates two intensely metabolically active nodal metastases, one in the right axilla and the other in the right upper anterior cervical triangle. These are thought to represent synchronous nodal metastases from two different cutaneous squamous cell carcinomas (previously resected). The CDI-PET (⁶⁸Ga NODAGA GSAO) demonstrates intense uptake in the right axillary nodal metastasis (SUVmean=4.1) and mild uptake in the right anterior cervical triangle nodal metastasis (SUVmean=1.7). The tumours were surgically excised, fixed and adjacent sections stained for apoptotic cells (FIG. 34C, brown TUNEL stain, a and b) or for morphology by haematoxylin and eosin (FIG. 34C, c and d). Arrows in the TUNEL staining point to areas of extensive apoptosis.

Note that those tumours with high uptake have uptake up to 2 fold greater than blood pool, and the uptake is greater than uptake in all other organs except for the renal tract which is the route of excretion. This high level of uptake within some tumours combined with the low level of activity within normal tissues and organs demonstrates the potential for use of ⁶⁸Ga NODAGA GSAO as an effective imaging agent.

### DISCUSSION

The interim analysis of the first four patients in this first in human study of ⁶⁸Ga-NODAGA-GSAO demonstrates it is safe, well-tolerated and without adverse effects. The biodistribution and imaging characteristics are favourable with only low levels of activity in most normal organs. The urinary tract is the only route of excretion. Uptake into dead and dying cells in the tumour is seen and ⁶⁸Ga-NODAGA-GSAO tumour uptake variable consistent with varying tumour histologies, and has been demonstrated histopathologically to correlate with the proportion of dead and dying cells within the tumour. The effective whole-body dose from 68Ga NODAGA GSAO ranged from 2.16×10−2 to 3.38×10−2 mSv/MBq, giving an estimated effective whole-body dose ranging from 4.3-6.8 mSv for ad administered activity of 200 MBq. This is comparable to many other diagnostic radiopharmaceuticals used for PET/CT and SPECT/CT as well as for effective whole-body dose from other radiologic procedures such as x-ray computed tomography (CT).

The disclosure of PCT application no. PCT/AU2020/050359 (published as WO2020206503) is incorporated herein by reference.

1. A compound according to Formula (I) 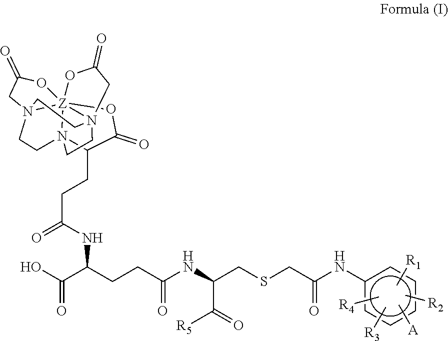
wherein A is —As(OH)₂ or an arsenoxide equivalent group;
each of R₁, R₂, R₃ and R₄ is independently selected from H, X, OH, NH₂, CO, SCN, —CH₂NH, —NHOOCH₃, —NHCOCH₂X or NO, and X is a halogen;
R₅ is —NHCH₂COOH, OH or OR₆, wherein Rₑ is a C₁₋₅ straight or branched alkyl group;
and Z is a therapeutic radioisotope,
or a pharmaceutically acceptable salt, ester, prodrug or solvate thereof.

2. The compound according to claim 1, wherein each of R₁, R₂, R₃ and R₄ are H.

3. The compound according to claim 1, wherein R₅ is —NHCH₂COOH.

4. The compound according to claim 1, which is a compound according to Formula (Ia) 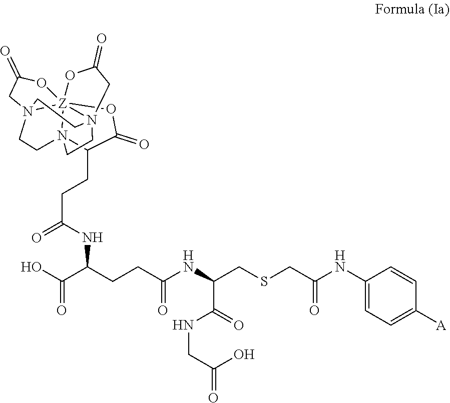
wherein A and Z are as defined in claim 1,
or a pharmaceutically acceptable salt, ester, prodrug or solvate thereof.

5. The compound according to claim 1, wherein Z is ¹⁷⁷Lu, ⁶⁴Cu, ⁶⁷Cu, ⁹⁰Y, ¹⁸⁶Re or ¹⁸⁸Re.

6. The compound according to claim 5, wherein Z is ¹⁷⁷Lu or ⁶⁷Cu.

7-13. (canceled)

14. A pharmaceutical composition comprising the compound of claim 1 together with a pharmaceutically acceptable carrier, excipient, diluent, vehicle and/or adjuvant.

15. A method of treating a neoplastic condition in a subject comprising administering an effective amount of a compound according to claim 1 to said subject.

16. The method according claim 15 wherein the neoplastic condition is a tumor.

17. The method according to claim 16 wherein the tumor is a solid tumor.

18. The method according to claim 15, wherein the neoplastic condition is cancer.

19. The method according to claim 15 wherein the compound is administered intravenously.

20. The method according to claim 17, comprising administering an effective amount of the compound to said subject in two or more cycles, wherein efficacy of the administration against the neoplastic condition increases across the two or more cycles.

21. The method according to claim 15 comprising:
a) carrying out a treatment for said neoplastic condition on the subject other than administering an effective amount of the compound to said subject; and
b) administering an effective amount of the compound to said subject.

22. The method according to claim 21, wherein the treatment carried out in step a) is chemotherapy, immunotherapy, radiotherapy and/or targeted therapy.

23. The method according to claim 21, wherein step a) is carried out concurrently with step b), or step b) is carried out after step a).

24. The method according to claim 21, wherein step b) is carried out for two or more cycles.

25. The method according to claim 24, wherein the efficacy of step b) against the neoplastic condition increases across the two or more cycles.

26. The method according to claim 24, wherein both steps a) and b) are carried out for two or more cycles.

27. The method according to claim 15, wherein the compound treats the neoplastic condition by inducing cell death.

28. A method of inducing cell death in a subject, comprising administering a compound according to claim 1 to a subject.

29. The method according to claim 28, wherein the compound is administered to a subject in multiple cycles, wherein the amount of cell death induced increases across the multiple cycles.

30-35. (canceled)

36. A process for preparing a compound according to claim 1, comprising adding the therapeutic radioisotope to a compound according to Formula (II) 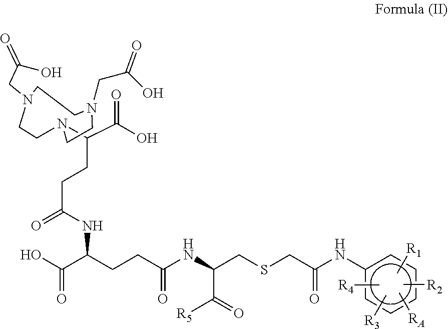
wherein A is —As(OH)₂ or an arsenoxide equivalent group;
each of R₁, R₂, R₃ and R₄ is independently selected from H, X, OH, NH₂, CO, SCN, —CH₂NH, —NHCOCH₃, —NHCOCH₂X or NO, and X is a halogen;
R₅ is —NHCH₂COOH, OH or OR₆, wherein R₆ is a C₁₋₅ straight or branched alkyl group;
or a pharmaceutically acceptable salt, ester, prodrug or solvate thereof.

37-49. (canceled)

50. A process for preparing a compound according to Formula (I) 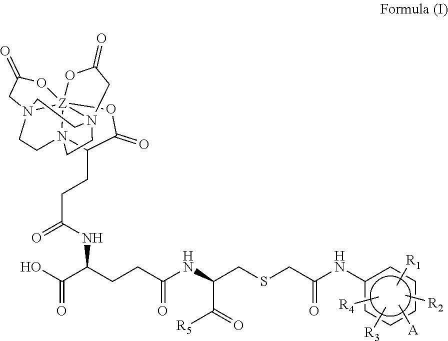
wherein A is —As(OH)₂ or an arsenoxide equivalent group;
each of R₁, R₂, R₃ and R₄ is independently selected from H, X, OH, NH₂, CO, SCN, —CH₂NH, —NHOOCH₃, —NHCOCH₂X or NO, and X is a halogen;
R₅ is —NHCH₂COOH, OH or OR₆, wherein R₆ is a C₁₋₅ straight or branched alkyl group;
and Z is a radioisotope,
or a pharmaceutically acceptable salt, ester, prodrug or solvate thereof,
said process comprising adding the radioisotope to a compound according to Formula (II) 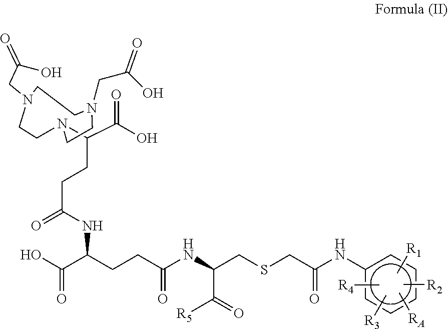
wherein A is —As(OH)₂ or an arsenoxide equivalent group;
each of R₁, R₂, R₃ and R₄ is independently selected from H, X, OH, NH₂, CO, SCN, —CH₂NH, —NHCOCH₃, —NHCOCH₂X or NO, and X is a halogen;
R₅ is —NHCH₂COOH, OH or OR₆, wherein R₆ is a C₁₋₅ straight or branched alkyl group;
or a pharmaceutically acceptable salt, ester, prodrug or solvate thereof, wherein the radioisotope is added to the compound of Formula (II) in the presence of glutathione.

51-56. (canceled)
